(19) United States
(12) Patent Application Publication
(10) Pub. No.: US 2024/0371476 A1
(43) Pub. Date: Nov. 7, 2024

(54) Vaccine Assessment and Compliance Testing Methods and Systems

(71) Applicant: Innovar Scientific, Inc., Denton, TX (US)

(72) Inventors: Richard A. Gilstrap, Denton, TX (US); Cantwell G. Carson, Pittsburgh, PA (US)

(21) Appl. No.: 18/683,950

(22) PCT Filed: Aug. 17, 2022
(86) PCT No.: PCT/US22/40612

§ 371 Date: Feb. 15, 2024

Related U.S. Application Data
(60) Provisional application No. 63/233,943, filed on Aug. 17, 2021.

(51) Int. Cl. G16C 20/30; G16H 20/10

(52) U.S. Cl. CPC G16C 20/30; G16H 20/10

(57) ABSTRACT

Various methods and corresponding systems for evaluating potency-correlated material states of a state dependent pharmaceutical product are disclosed. The method may include the steps of creating a data representation of material state specifications for a pharmaceutical product using data gathered from at least one sensor. The method may include correlating a minimum viable potency of the pharmaceutical product and communicating the data representation to at least one participant of a supply chain. The method may include the steps of generating a specimen representation of a material state of a sample of the pharmaceutical product using data gathered from at least one sensor acting on the sample and evaluating the specimen representation of the material state of the sample. The method may include the step of determining whether the specimen representation of the material state of the sample exhibits a material state change greater than the maximum allowable material state change.

### CROSS-REFERENCE TO RELATED APPLICATIONS

This application claims priority under 35 U.S.C. § 371 to PCT/US2022/040612 filed Aug. 17, 2022 which claims priority to U.S. Provisional Application No. 63/233,943, titled Quantitative Vaccine Assessment and Compliance, filed Aug. 17, 2021, the disclosure of each is incorporated herein by reference in entirety.

### FIELD

The present technology is generally related to a method and system for analyzing the potency of a pharmaceutical product such as a vaccine.

### BACKGROUND

The safety and effectiveness of many pharmaceuticals is dependent upon one or more specified material states. In the case of vaccines, this is true of both material constituents and final products. For example, the potency of an adsorbed vaccine depends in part, upon the proper charge state of its adjuvants, conformational character of its antigens, and structural stability of the coordinated adjuvant-antigen phase. Thus, a change in the state of these pharmaceutical products may directly undermine their safety and effectiveness towards human and animal health. Due to their complex architecture, nanoscale dimensions, and sensitive biological nature, a wide variety of vaccines require stringent handling practices to maintain their specified material state and associated potency. A well-documented failure to maintain such practices throughout the pharmaceutical supply-chain has motivated the development and adoption of various environmental monitoring technologies. In contrast, relatively little effort has been focused on developing techniques to evaluate the actual condition of vaccines within the global supply-chain.

### SUMMARY

The techniques of this disclosure generally relate to methods, systems, and an apparatus for evaluating potency-determining and/or potency-correlated material states of a state dependent pharmaceutical product such as a vaccine.

In various embodiments, a method for evaluating potency-determining and/or potency-correlated material states of a state dependent pharmaceutical product, is disclosed. The method may include the steps of creating a data representation including a plurality of material state specifications for a pharmaceutical product using data gathered from at least one sensor acting on the pharmaceutical product, the material state specifications including a maximum allowable material state change. The method may include the steps of correlating a minimum viable potency of the pharmaceutical product to the maximum allowable material state change and communicating the data representation to at least one participant of a supply chain of the pharmaceutical product. The method may include the steps of generating a specimen representation of a material state of a sample of the pharmaceutical product using data gathered from at least one sensor acting on the pharmaceutical product sample and evaluating the specimen representation of the material state of the sample. The method may also include the step of determining whether the specimen representation of the material state of the sample exhibits a material state change greater than the maximum allowable material state change of the material state specifications.

In various embodiments, the method may include discarding at least one pharmaceutical product if the specimen representation of the material state of the sample exhibits a material state change greater than the maximum allowable material state change of the material state specifications. In some embodiments, the at least one pharmaceutical product may be associated with a corresponding localized supply of the sample of the pharmaceutical product.

In various embodiments, the method may include communicating identification data of the at least one pharmaceutical product to be discarded to at least one participant of the supply chain of the pharmaceutical product.

In various embodiments, the method may include accepting at least one pharmaceutical product for use if the specimen representation of the material state of the sample exhibits a material state change less than the maximum allowable material state change of the material state specifications. In at least some embodiments, the at least one pharmaceutical product for use is associated with a corresponding localized supply of the sample of the pharmaceutical product.

In various embodiments, the method may include communicating identification data of the at least one pharmaceutical product to at least one participant of the supply chain of the pharmaceutical product.

In various embodiments, the method may include determining an estimated potency of the sample of the pharmaceutical product; and communicating the estimated potency of the sample to at least one participant of the supply chain.

In some embodiments, the evaluating the specimen representation of the material state of the sample step further includes inputting at least one data component of the specimen representation of the material state of the sample of the pharmaceutical product into a neural network. In at least some embodiments, the neural network comprises a trained machine learning model.

In various embodiments, the method may include categorizing, by the trained machine learning model, whether the specimen representation of the material state of the sample comprises an acceptable material state change or an unacceptable material state change.

In various embodiments, the method may include training the machine learning model based on the data representation of the plurality of material state specifications for the pharmaceutical product.

In various embodiments, the method may include communicating, at a local level, at least one communication indicative of whether (1) the specimen representation of the material state of the sample exhibits a material state change determined to be greater than the maximum allowable material state change of the material state specifications or (2) the specimen representation of the material state of the sample exhibits a material state change determined to be less than the maximum allowable material state change of the material state specifications. In at least some embodiments, the communication is chosen from the group comprising: a visual communication, an auditory communication, and/or a haptic communication.

In various embodiments, the method may include communicating, across at least one blockchain network, at least one communication indicative of whether (1) the specimen representation of the material state of the sample exhibits a material state change determined to be greater than the maximum allowable material state change of the material state specifications or (2) the specimen representation of the material state of the sample exhibits a material state change determined to be less than the maximum allowable material state change of the material state specifications. In at least some embodiments, the communication includes data corresponding to an evaluation outcome of the evaluating the specimen representation of the material state of the sample step.

In at least some embodiments, the at least one blockchain network is accessible by a plurality of different participants of the supply chain of the pharmaceutical product.

In at least some embodiments, the at least one blockchain network provides a permanent ledger for performing an audit.

In at least some embodiments, the at least one blockchain network is only accessible by a subset of permissioned participants of the supply chain of the pharmaceutical product.

In at least some embodiments, the evaluating the specimen representation of the material state of the sample step further includes evaluating the extent of chemical or conformational change in the pharmaceutical product.

In at least some embodiments, the evaluating the specimen representation of the material state of the sample step further includes evaluating the extent of phase separation in the pharmaceutical product.

In at least some embodiments, the phase separation is chosen from the group including: agglomeration, flocculation, coalescence, creaming, and/or Ostwald ripening.

In at least some embodiments, the pharmaceutical product is a vaccine developed for human and/or animal use.

In at least some embodiments, the evaluating the specimen representation of the material state of the sample step further comprises evaluating the extent of change in the conformation of antigens.

In at least some embodiments, the pharmaceutical product is a vaccine comprising an adjuvant.

In at least some embodiments, the evaluating the specimen representation of the material state of the sample step further comprises evaluating the extent of phase separation of the coordinated antigen-to-adjuvant vaccine components.

In at least some embodiments, the evaluating the specimen representation of the material state of the sample step further comprises evaluating the extent of phase separation of the non-coordinated adjuvant vaccine component.

In at least some embodiments, the adjuvant comprises an oil and water emulsion.

In at least some embodiments, the pharmaceutical product is a vaccine component material.

In at least some embodiments, the pharmaceutical product is a vaccine component comprising an adjuvant.

In at least some embodiments, the creating the data representation including the plurality of material state specifications for a pharmaceutical product step further includes utilizing a phase contrast microscopy process to evaluate a size of the agglomerates.

In at least some embodiments, the evaluating the specimen representation of the material state of the sample further includes evaluating a size of the agglomerates.

In at least some embodiments, the at least one sensor includes a 2-D optical detector.

In various embodiments, an apparatus for evaluating potency-correlated material states of a state dependent pharmaceutical product is disclosed. The apparatus may include a housing having a receptacle for receiving a sample of a state dependent pharmaceutical product; a light emitting diode (LED) configured to emit light through the receptacle and the sample of the state dependent pharmaceutical product; and at least one 2-D optical detector configured to receive the emit light after it has passed through the sample of the state dependent pharmaceutical product. The apparatus may further include a controller configured to receive optical data from the at least one 2-D optical detector; create a sample specific photonic profile based on the received optical data of the sample of the state dependent pharmaceutical product; and compare the sample specific photonic profile to a pre-established photonic specification profile, the pre-established photonic specification profile comprising a photonic profile of a viable state of the state dependent pharmaceutical product.

In various embodiments, the controller may be further configured to determine whether the sample specific photonic profile is indicative of a non-viable product by assessing whether a data point of the sample specific photonic profile is outside of an upper boundary value and/or a lower boundary value of the pre-established photonic specification profile.

In various embodiments, the state dependent pharmaceutical product is a vaccine developed for human and/or animal use.

In various embodiments, the state dependent pharmaceutical product is a vaccine comprising at least one adjuvant; and the sample specific photonic profile is indicative of a degree of phase separation of the coordinated antigen-to-adjuvant vaccine components, a degree of phase separation of the non-coordinated adjuvant vaccine components, a degree of vaccine component dispersion, a degree of conglomeration, and/or a degree of settlement of the at least one adjuvant.

In another aspect, a system for evaluating potency-correlated material states of a state dependent pharmaceutical product is disclosed. The system may include a processor in communication with at least one non-transitory memory cell storing computer executable instructions thereon that when executed by the processor are configured to perform a series of operations. The processor may generate a data representation comprising a plurality of material state specifications for a pharmaceutical product using data gathered from at least one sensor acting on the pharmaceutical product, the material state specifications including a maximum allowable material state change. The processor may correlate a minimum viable potency of the pharmaceutical product to the maximum allowable material state change, communicate the data representation to at least one participant of a supply chain of the pharmaceutical product, and receive a data representation comprising a plurality of material state specifications for a pharmaceutical product, the material state specifications including a maximum allowable material state change. The processor may generate a specimen representation of a material state of a sample of the pharmaceutical product using data gathered from at least one sensor acting on the pharmaceutical product sample, and utilize a machine learning model to evaluate the specimen representation of the material state of the sample in order to determine whether the specimen representation of the material state of the sample exhibits a material state change greater than the maximum allowable material state change of the material state specifications. The processor may communicate the specimen representation of the material state of the sample and its evaluation to at least one participant of a supply chain of the pharmaceutical product.

In another aspect, a computer program product is disclosed. The computer program product may be embodied in a non-transitory computer readable storage medium and include computer executable instructions. The instructions may include generating a data representation comprising a plurality of material state specifications for a pharmaceutical product using data gathered from at least one sensor acting on the pharmaceutical product, the material state specifications including a maximum allowable material state change. The instructions may include correlating a minimum viable potency of the pharmaceutical product to the maximum allowable material state change, and communicating the data representation to at least one participant of a supply chain of the pharmaceutical product. The instructions may include receiving a data representation comprising a plurality of material state specifications for a pharmaceutical product, the material state specifications including a maximum allowable material state change, and generating a specimen representation of a material state of a sample of the pharmaceutical product using data gathered from at least one sensor acting on the pharmaceutical product sample. The instructions may include utilizing a machine learning model to evaluate the specimen representation of the material state of the sample in order to determine whether the specimen representation of the material state of the sample exhibits a material state change greater than the maximum allowable material state change of the material state specifications. The instructions may include communicating the specimen representation of the material state of the sample and its evaluation to at least one participant of a supply chain of the pharmaceutical product.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 illustrates an example relationship between material state on vaccine potency and a measurable property directly associated with that state.

FIG. 2 illustrates a baseline model heuristic of state change evaluation in vaccines.

FIG. 3 illustrates a baseline model heuristic of state change evaluation in vaccines which includes allowable threshold parameters.

FIG. 4 illustrates a flow diagram illustrating an embodiment of a process for assessing vaccines for compliance with material state specifications.

FIG. 5 illustrates an increased settling rate that indicates the incidence of phase separation and associated potency reduction.

FIG. 6 illustrates a threshold model heuristic of freeze-damage evaluation in adsorbed adjuvant vaccines.

FIG. 7 illustrates sedimentation profiles produced from three hypothetical 1-D light detector positions near the top, middle, and bottom of an adsorbed vaccine liquid suspension.

FIG. 8 illustrates application of a 2-D optical sensor to the evaluation of an unopened vial of adjuvanted vaccine which produces a 3-D photonic profile that displays the temporal progression of transmitted light versus vial position.

FIG. 9 illustrates photonic profiles of single-dose presentations of pentavalent vaccine in as-specified and freeze-damaged conditions.

FIG. 10 is a flow diagram illustrating an embodiment of a process for training and applying a machine learning model to evaluate specimen representation data.

FIG. 11 is an example of a blockchain implementation of the system and methods described herein.

FIG. 12 is an example Venn diagram presenting an embodiment for a complete system and method that incorporates vaccine data generation, machine learning evaluation, and blockchain encryption/communication.

FIG. 13 is an example embodiment of localized and distributed functional systems for applying the baseline model heuristic to vaccine evaluation.

FIG. 14 is an example flow chart illustrating an embodiment of a localized system.

FIG. 15 is an example flow chart illustrating how a distributed system may operate.

### DETAILED DESCRIPTION

The following discussion omits or only briefly describes certain components, features and functionality related to vaccine preparation, vaccine assessment, monitoring techniques, quantitative analysis, computer hardware, blockchain technologies, and associated hardware which are apparent to those of ordinary skill in the art. It is noted that various embodiments are described in detail with reference to the drawings, in which like reference numerals represent like parts and assemblies throughout the several views, where possible. Reference to any of the various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Embodiments in accordance with this disclosure can be implemented in numerous ways, including as a method; a process; a system; an apparatus; a composition of matter; a computer program product embodied on a computer readable storage medium or programable processor; a processor and/or controller, such as a processor configured to execute instructions stored on and/or provided by a memory coupled to the processor. Unless stated otherwise, a component such as a sensor, a processor, or a memory described as being configured to perform a task may be implemented as a general component that is temporarily configured to perform the task at a given time or a specific component that is manufactured to perform the task.

As used herein, the term “processor” refers to one or more devices, circuits, and/or processing cores configured to process data, such as computer program instructions. Furthermore, the term “controller” may, for example, refer to a processor and include one or more of the following components: at least one central processing unit (CPU) configured to execute computer program instructions to perform various processes and methods, random access memory (RAM) and read only memory (ROM) configured to access and store data and information and computer program instructions, input/output (I/O) devices configured to provide input and/or output to the processing controller (e.g., keyboard, mouse, display, speakers, printers, modems, network cards, etc.), and storage media or other suitable type of memory (e.g., such as, for example, RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives, any type of tangible and non-transitory storage medium) where data and/or instructions can be stored. In addition, the controller can include antennas, network interfaces that provide wireless and/or wire line digital and/or analog interface to one or more networks over one or more network connections (not shown), a power source that provides an appropriate alternating current (AC) or direct current (DC) to power one or more components of the controller, and a bus that allows communication among the various disclosed components of the controller.

In this specification, these implementations, or any other form that any embodiment herein may take, may be referred to as techniques and/or methods. In general, the order of the steps of disclosed processes may be altered and remain within the scope of the disclosure. Furthermore, methods of use need not necessarily be performed in any particular order. Hereinafter, some embodiments of the present disclosure will be described in detail with reference to the drawings.

Disclosed herein are a system and method to assess vaccines for compliance with material state specifications. While the safety and effectiveness of a broad variety of pharmaceutical products rely only on composition, many are also dependent upon one or more distinct material states (i.e., chemical, electrical, structural, etc.). This application may refer to this broad category of materials as State-Dependent Pharmaceutical Products (SDPPs) with vaccines being a prominent example. As used in this specification, the term “State-Dependent Pharmaceutical Product” may refer to any pharmaceutical product and/or supplement whose safety and/or effectiveness is dependent upon one or more distinct material states. Furthermore, SDPPs may broadly refer to any product that may be injected and/or ingested into a body of a human or animal. In some instances, SDPPs may refer to the broad class of patient specific treatments which are designed and/or tailored in mind of a specific patient, e.g., the specific patient's genetics.

At least one example SDPP may be a vaccine. Vaccines are biological compositions that are state-dependent pharmaceutical products which illicit a targeted immune response to specific pathogens and/or other cell types of interest. The efficacy or potency of an immune response to the vaccine is critically dependent upon a variety of specified vaccine material states such as, but not limited to, structure. As part of the development process, a vaccine's material states are optimized to produce the highest degree of efficacy and potency possible. For example, a physical, biological, and/or chemical structure of the vaccine is optimized, within reason, for the purpose of eliciting a strong immune response of the future patient. Accordingly, any deviation from these optimized states typically results in potency reduction.

Due to the instability of biological structures, the potency of vaccines is observed to degrade as a function of time and the environmental conditions to which they are exposed. In many jurisdictions, the expiration date printed on a given vial or other container of a vaccine is explicitly valid only if its contents have been maintained within a very narrow temperature range from which stability data was historically derived. A common temperature range is typically about 2° C. to about 8° C. for liquid vaccine versions. Other vaccines may require storage and handling temperatures as low as −70° C. in order to prevent premature material state change of the vaccine. In all state dependent vaccines, exposure to temperatures outside their specified handling range will cause material state changes to the vaccine and thereby directly reduce potency. Consequently, more than 90% of all vaccines require a temperature-controlled supply chain which may be referred to as the “cold chain” in the relevant industry. In common parlance, the “cold chain” begins at the point of origin, i.e., the manufacturers facility and throughout the entirety of the vaccines' transportation across each and every location along the “cold chain”, the vaccine must be maintained in accordance with the manufacturers' specifications until the vaccine is administered to the patient.

Excursions above the manufacturers' specified temperature range typically reduce potency through protein denaturing and conformational changes on specific proteins and antigens. In some instances, these high temperature induced changes may be gradual. For example, with the exception of a temperature excursion in excess of 50° C., the high-temperature-induced change may be relatively gradual in nature, occurring over the course of about 2-30 days depending upon the particular vaccine type. In the case of fully liquid vaccines, excursions below the 2° C.-8° C. range into freezing temperatures may result in severe structural change, e.g., freeze-damage, which abruptly reduces, or entirely eliminates, potency. For example, when undergoing phase change the physical structure of a vaccine may be irreparably altered. Furthermore, it can be very difficult to know whether a vaccine has been exposed to temperatures above and/or below the manufacturers specified temperature range. For example, unless a vial or other container of vaccine is found and visually observed to be frozen solid, a participant of the supply chain is typically unable to realistically detect such incidences of low-temperature-induced material state change. Depending on usage, the phrase “state change” refers to a material state change of a pharmaceutical product and may refer to a material state change of a vaccine unless the context clearly indicates otherwise.

Although many of the vaccines most critical to worldwide disease prevention are extremely temperature-sensitive, the incidence of both high and low temperature excursions throughout the pharmaceutical cold chain is significant. For example, a landmark 2007 study by the global health organization, PATH, highlighted the extent of this problem on a worldwide scale. They found that exposure of vaccines to freezing temperatures was pervasive, occurring in both developed and developing-country settings, within both storage and transport segments of the cold chain. Across the four scenarios analyzed, the average proportion of exposure ranged from 14% to 35% and in the six studies that measured temperatures longitudinally, through multiple sections of the cold chain, between 75% and 100% of the vaccine shipments were exposed to freezing temperatures. Although this study helped to motivate the expanded use of continual temperature data logging devices, a subsequent 2017 update found that accidental freezing exposure had further increased over the previous decade as the pharmaceutical cold chain grew in both size and complexity. It is important to note that the accuracy resolution of temperature data loggers is limited to the region immediately adjacent to and/or very near such devices. Accordingly, a significantly wide temperature variation may exist within a given storage volume which readily produces potency-reducing vaccine state change.

As exposure to temperature excursions outside manufacturer specifications remains an ongoing issue, a need exists for accurate vial-level evaluation methods to ensure that vaccines have retained their specified state and associated properties prior to patient administration. Furthermore, these methods must be practical for use throughout all sections of the pharmaceutical cold chain. No rapid physiochemical assays are currently available to directly measure vaccine potency and due to their macromolecular complexity, there exist no simple correlations between a given temperature excursion and specific potency reduction. However, correlations can be made between specific state changes of a SDPP and the consequent potency reduction thereof. Vaccine material state is highly optimized for potency, and therefore, any change in this optimized state may be directly correlated with potency reduction. The technology described herein allows one to evaluate these potency-correlated states in unopened containers of vaccine through an analysis of their directly correlated measurable properties.

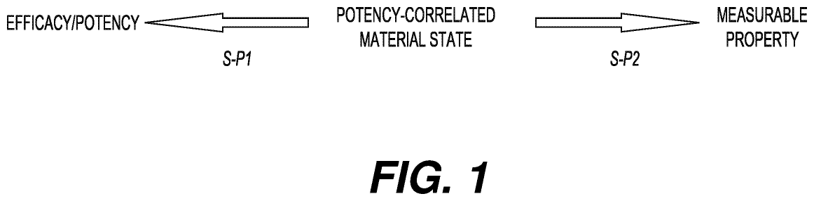

Referring to FIG. 1, the systems and methods described herein are based upon the development of a baseline conceptual model in which the influence of vaccine material state is correlated along two State-Property (S-P) relations. S-P1 may refer to the relationship between material state and vaccine potency and S-P2 may refer to the relationship between material state and one or more measurable properties that are directly associated with that state. Various example measurable properties may include settling behavior, turbidity, and creaming which are discussed in further detail below. As will be discussed in further detail below, identifying both the absence of a vaccine state change and the incidence of a vaccine state change may inform such an approach.

In various embodiments, by applying sensors to measure one or more such properties of an SDPP, e.g., (electrical, chemical, acoustic, electromagnetic, structural, etc.), a “data-representation” of a plurality of potency-correlated states may be generated and used for subsequent vaccine assessment. For example, a vaccine that has just been or has been recently manufactured and is known to have a high efficacy may undergo various types of testing to build a “data-representation” of the SDPP as a control. In various embodiments, sampling of a plurality of vaccines that are known to have a high potency/efficacy may be independently tested and compiled together as an average profile and/or an “average data-representation” to assist with small deviations that may occur due to manufacturing inconsistencies, e.g. In at least some embodiments, a manufacturer may utilize high quality sensors to perform this testing which are not necessarily readily available and/or practical to use in the field and/or further down the supply chain. Due to the lack of field specimens known to be in specified condition, compiling vaccine data from known high quality control samples may be required for an accurate evaluation and/or creation of a “data-representation.”

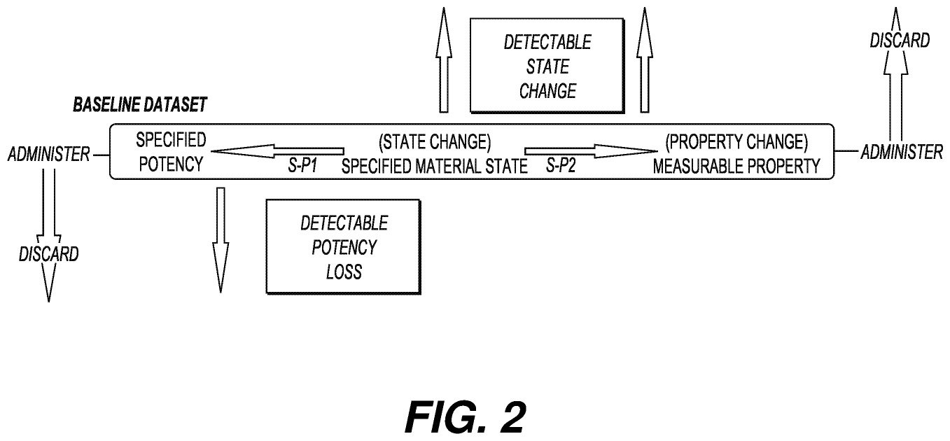

Referring to FIG. 2, a Baseline Model heuristic may be shown. As seen in FIG. 2, in this model, the baseline represents a threshold (e.g., a lower threshold) for the incidence of some event and in the present example, that event is a potency-correlated state change. In other embodiments, a model may further conceptualize the baseline of FIG. 2 as an upper threshold corresponding to the absence of that particular event. For any given vaccine category, measurements may be applied to and/or acquired with respect to a representative sample of an SDPP, or samples (independently verified to be characterized by the desired or specified state), in order to create a baseline dataset, under strictly controlled and optimal conditions. The dataset is comprised of a specified potency (or potency of interest) correlated to a specified material state (or state of interest) and one or more measurable properties that are directly associated with that state, e.g., the “Specified Representation” and/or “SDPP Specifications.” In various embodiments, this Specified Representation may comprise the primary data against which all subsequent measurements of specimen vaccines from that category are evaluated. For example, a participant later down the supply chain may compare a sample of the SDPP to the “specified representation” and/or “SDPP Specifications.” If the measured properties of a sample specimen SDPP (the Specimen Representation), selected for testing, are determined to comply with those defined by its Specified Representation, that vaccine is determined to be “As-Specified” and deemed safe and effective for patient administration. Otherwise, it is determined to be out of specifications and discarded. Furthermore, in this way, a participant later down the supply chain can also tell whether an SDPP has been tampered with and/or may be contaminated by comparing the material state data of the Specimen Representation against the “SDPP Specifications.

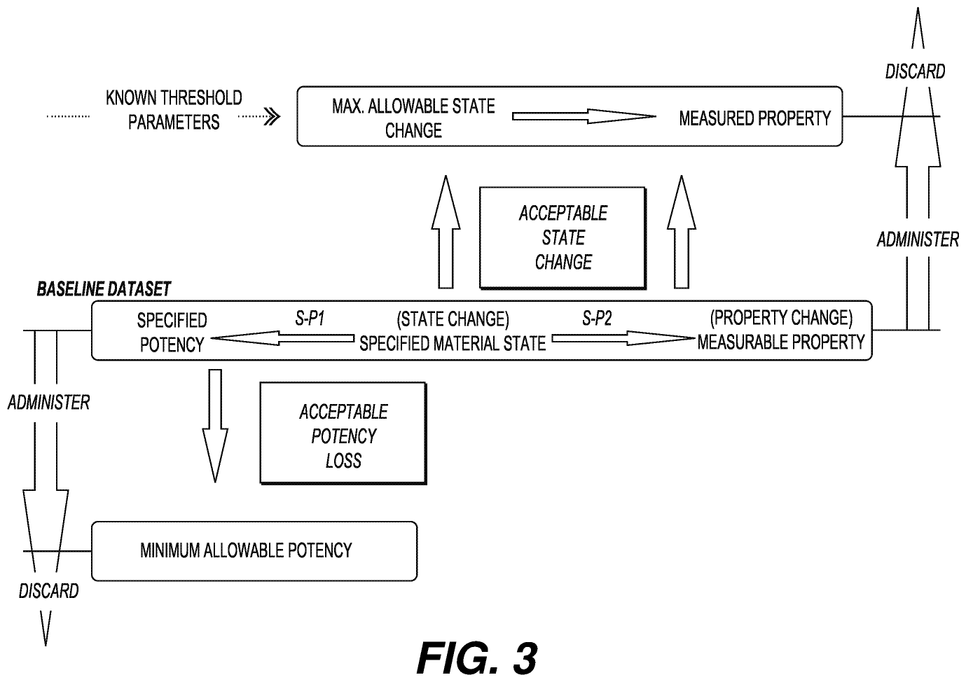

Referring generally to FIG. 3, a Baseline Model Heuristic of state change evaluation of an SDPP may include allowable threshold parameters. In various embodiments, and where applicable, the baseline dataset and associated Specified Representation may be augmented to include a minimum allowable potency, which may then be correlated to a maximum allowable extent of state change.

### Example Working Technique 1

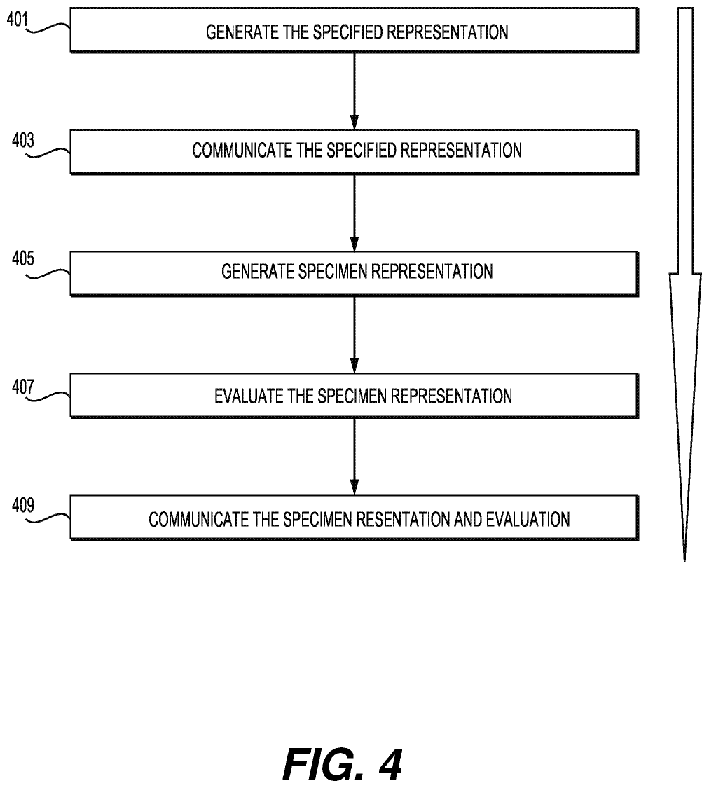

In general, a technique to assess SDPPs, such as vaccines, for compliance with material state specifications is disclosed. This technique may be performed using one or more analytical devices for testing material properties of the SDPP. Such devices may be incorporated within a variety of vaccine storage, handling, and/or administration systems (e.g., containers, vials, syringes, etc.). For any given SDPP type and/or category, a data-representation of its specified material state is generated and subsequently utilized in the evaluation of sample specimens from that same category. This technique or system may include some or all the elements described below and illustrated in the flow chart of FIG. 4.

Referring generally to FIG. 4, a flowchart of a first technique for evaluating a sample of an SDPP, in this case a vaccine, against the specifications of the SDPP is shown. First, the system may define the data-representation of a specified vaccine state (the Specified Representation). At step 401, the Specified Representation may be generated. However, it shall be appreciated that in many instances the Specified Representation may be already known, e.g., at a later point in time. In some embodiments, this is accomplished by applying a variety of sensors (electrical, chemical, acoustic, optical, etc.) to vaccine samples of known material state similarly as explained above. When that known state is the specified or desirable state, these measurements constitute the specified data-representation for that potency-correlated vaccine state, e.g., a control and/or known good vaccine. The potency-correlated states of vaccines vary widely depending upon their specific type and therefore, some embodiments require that a wide variety of sensor measurements be applied. Accordingly, those with skill in the art will readily appreciate that the specific sensor measurements may vary according to the type of SDPP at issue. This Specified Representation comprises a core component of the Baseline Dataset previously described above.

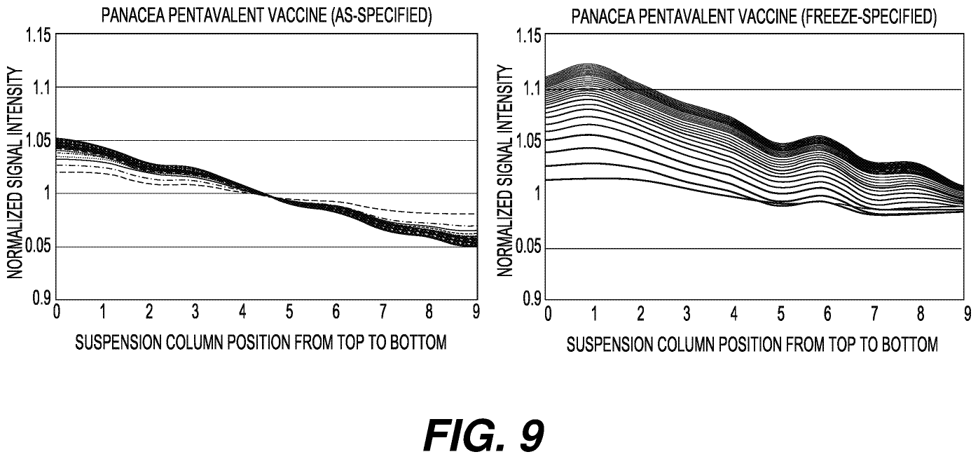

Once the specified representation is known, the system may communicate the specified representation publicly across the supply chain and/or privately to those participants who have the appropriate permissions and/or credentials. For example, at step 403, the Specified Representation is communicated. When a shipment of vaccine has been received at some point within the pharmaceutical cold-chain, none of the containers are truly known to be in accordance with the as-specified condition (e.g., there are no “known-good examples” with which to compare specimen vaccines). Accordingly, the data-representation of a known-good vaccine (the Specified Representation) may be made available throughout the pharmaceutical cold-chain using a variety of techniques. In some embodiments, this communication may be accomplished by uploading the Specified Representation to a Blockchain ledger system that provides both data encryption/security and a permanent audit trail. In some embodiments, the Blockchain ledger may be a public ledger, e.g., the Ethereum Network, the Solana Network, the Avalanche Network, Cardano Network, Binance Smart Chain Network to name a few examples. In other embodiments, the communication may be as simple as an email having a corresponding data set. In other embodiments still, the communication may simply be stored in a memory of a device, e.g., the device discussed in FIG. 8 and such memory of the device may be updated from time to time for subsequent variations in the SDPP.

At step 405, the Specimen Representation is generated. For example, the system may generate the data-representation of an SDPP specimen state (the Specimen Representation). In some embodiments, this is accomplished by applying similar sensors to a specimen of an SDPP (e.g., a vaccine) as those utilized for the Specified Representation, such that measurement equivalence is ensured. For example, when an optical detector is used to gather material state data to generate the material state specifications of a control SDPP, a device with similar hardware which operates along similar principles and utilizes similar settings may be helpful to ensure measurement equivalence.

At step 407, the Specimen Representation may be evaluated. For example, the system may evaluate the SDPP specimen with respect to its specifications. This step may include comparing the Specimen Representation to the applicable Specified Representation to determine if the sample of the SDPP (e.g., the vaccine) is within the material state specifications. In some embodiments, this evaluation is accomplished using machine learning methods. For example, data comprising the Specimen Representation may be fed as an input and/or a plurality of inputs to a neural network using a trained machine learning model. In various embodiments, the trained machine learning method may categorize the specimen as either excellent (almost no material state change and/or insignificant material state change), good (an acceptable material state change within acceptable guidelines) and/or bad (unacceptable material state change indicating not to use the SDPP). In some embodiments, the machine learning model is trained with data comprising the Specified Representation.

At step 409, the Specimen Representation and/or its evaluation may be communicated. For example, the system may communicate the specimen representation (sample SDPP material state) and/or the evaluation results. In various embodiments, a local communication and/or a supply chain wide communication, or a targeted communication may be performed. In some embodiments, a machine (see e.g., FIG. 8) may generate a local communication. Such a local communication may be accomplished by visual, auditory, or haptic means that are processed by a system or hardware testing machine for validating SDPP material states of a sample in the field and/or later down the supply chain. These communications may tell a participant of the supply chain at the point of testing that the vaccines are not acceptable for use. For example, a red light of the machine may indicate an SDPP is bad whereas a green light may indicate the SDPP is within acceptable tolerances and/or approved for use. A supply chain wide/program wide communication may be accomplished by the machine by automatically transmitting a communication at large across a supply chain network. In this embodiment, the machine may be similar to a node of a larger interconnected supply chain network. For example, the communication transmitted by the machine may comprise a data upload to a variety of network or storage destinations. In some embodiments, this is accomplished by uploading the Specimen Representation and its evaluation to a Blockchain ledger system that provides both data encryption/security and a permanent audit trail, e.g., the Ethereum Network, the Solana Network, the Avalanche Network, Cardano Network, Binance Smart Chain Network to name a few examples.

Through the duration of various embodiments of a plurality of nodes/machines, SDPP material state data is generated, evaluated, and communicated on an ongoing and iterative process. In this way, the amount of available data for training a machine learning model for statistic analysis may always be growing and becoming more and more accurate over time. For example, application of the Baseline Model Heuristic and each of these data-management actions is further discussed below with the aid of an example SDPP category, in this case different types of vaccines, to which the data may be applied, re-applied, re-evaluated, and updated. In this sense, each node/machine may also store a plurality of data sets as source code/firmware for automatically evaluating existing and new SDPP that are later arising. It shall be appreciated that each example category does not limit the scope of disclosed technology as the techniques and methods described herein are fundamentally applicable to all SDPP, including vaccines, and whose potency-correlated or efficacy-correlated states are amenable to measurement.

### Example Working Technique 2: Adjuvant Vaccines

The relationship between state change and potency reduction may be of particular importance to vaccines which employ an adjuvant. Adjuvants provide an improved immune response by enhancing antigen affinity to antigen-specific immune cells. This is particularly beneficial for vulnerable populations such as infants, elderly, immunocompromised, and chronically ill patients. Use of appropriate adjuvants decreases the amount of antigen contained in each vaccine (antigen sparing), thus reducing the number of vaccine doses required to achieve sufficient protection (dose sparing). Overall, adjuvants improve sustainability of the global vaccine supply by reducing the need for additional clinic visits while also enhancing the effectiveness of vaccination rates and thus increasing the consequent herd immunity.

Importantly, the potency of an adjuvanted vaccine may be directly correlated with the stability of its material state, e.g., maintaining a vaccine adjuvant in the same material state as when it is immediately finished being manufactured. The majority of adjuvanted vaccines utilize a coordinated phase structure wherein antigens are adsorbed to the adjuvant primarily through hydrophobic, electrostatic and ligand exchange mechanisms. When such vaccines are exposed to freezing conditions of sufficient severity, uncoupling of these coordinated structures into separate antigen and adjuvant phases may occur. This “phase separation” presents a severe state change (freeze-damage) that causes abrupt potency reduction as previously described. Similar state changes may also occur in non-coordinated vaccine systems that utilize oil and water adjuvant emulsions (i.e., o/w, w/o, w/o/w, o/w/o, etc.). Likewise, similar state changes may also occur in supplements and other types of SDPP that utilize an oil and water emulsion.

This may be primarily due to differences in the phase transformation rates of oil and water which drives various phase separation mechanisms such as creaming, flocculation, coalescence, and Ostwald Ripening. Both the incidence and extent of phase separation in adjuvanted vaccines may be dependent upon compound specific material parameters and specific sample exposure parameters. In various embodiments, material state parameters may include (1) vaccine type, (2) manufacturer, (3) lot number, and (4) quantity of doses per unit (e.g., doses per vial). A fact specific set of these four parameters is often referred to as the “vaccine presentation” although one might imagine any number of additional core parameters and/or secondary parameters for effective tracing along a supply chain. In various embodiments, exposure parameters may include (1) temperature, (2) time, and/or (3) number of freeze-thaw cycles. For example, a given set of exposure parameters may severely damage a one-dose vial of a Pentavalent vaccine from Manufacturer A, while causing relatively minor damage to a ten-dose vial of HepB vaccine from Manufacturer B. Therefore, in this sense merely monitoring just the environmental conditions to which vials of vaccine are exposed is often an insufficient strategy for identifying actual freeze-damage and associated potency reduction in at least some SDPP. Pentavalent vaccine is but one example SDPP exhibiting this property. In another scenario, two freeze-thaw cycles (12 hours at −10° C. and 12 hours at −20° C.) may impart a greater degree of phase separation in the above HepB presentation than a single freeze-thaw cycle of 12 hours at −10° C. This second scenario helps illustrate why freeze-damage in adjuvanted vaccines is a complex and progressive phenomenon requiring accurate material evaluation at the individual vial level that can ascertain the viability of a specific sample by a participant later down the supply chain. Simply stated, the last person along the supply chain has no way of truly knowing whether the sample is good, and as illustrated above, different SDPP have associated individual difficulties with maintaining potency which can be easily violated along the supply chain by an upstream participant without the downstream participants' knowledge.

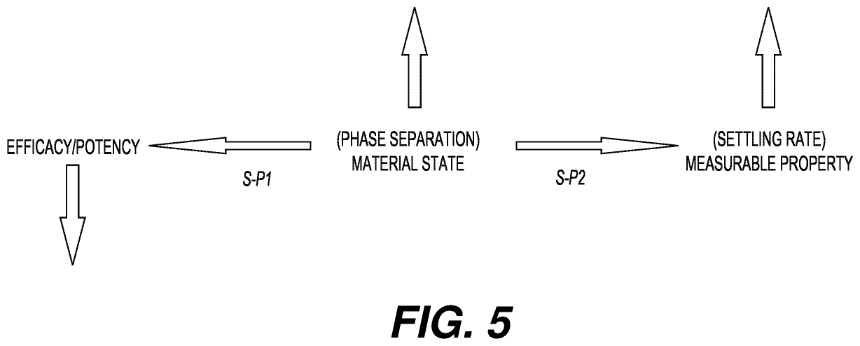

As discussed above, evaluating vaccines for evidence of state change may be accomplished by identifying measurable properties directly associated with its incidence and extent. For example, phase separation in adsorbed vaccines may create large adjuvant agglomerates that may be visible by the appropriate sensors and/or lens, e.g., large adjuvant agglomerates may be visible under phase contrast microscopy. Due to their greater mass, these agglomerates may substantially alter the particle settling characteristics of a freeze-damaged adsorbed vaccine. Accordingly, an increased settling rate directly indicates the incidence of phase separation (in this scenario freeze-damage) and associated potency reduction (see FIG. 5). Additionally, as will be explained in further detail below, these types of vaccine settling characteristics may be amenable to optical analysis. An example analysis according to optical methods may be described below. The below example describes an Adsorbed Vaccine, whose Potency-Correlated State is represented by a first variable, i.e., Phase Separation. In this way, the material state change may be surmised by a system and method that can evaluate, by measuring the associated particle settling characteristics, a material state change that may have occurred upstream along the supply chain. With reference back to FIG. 5, an illustration showing how phase separation produces adjuvant agglomerates that in turn increase the settling rate of adsorbed vaccines. This phase separation is directly correlated with abrupt potency reduction.

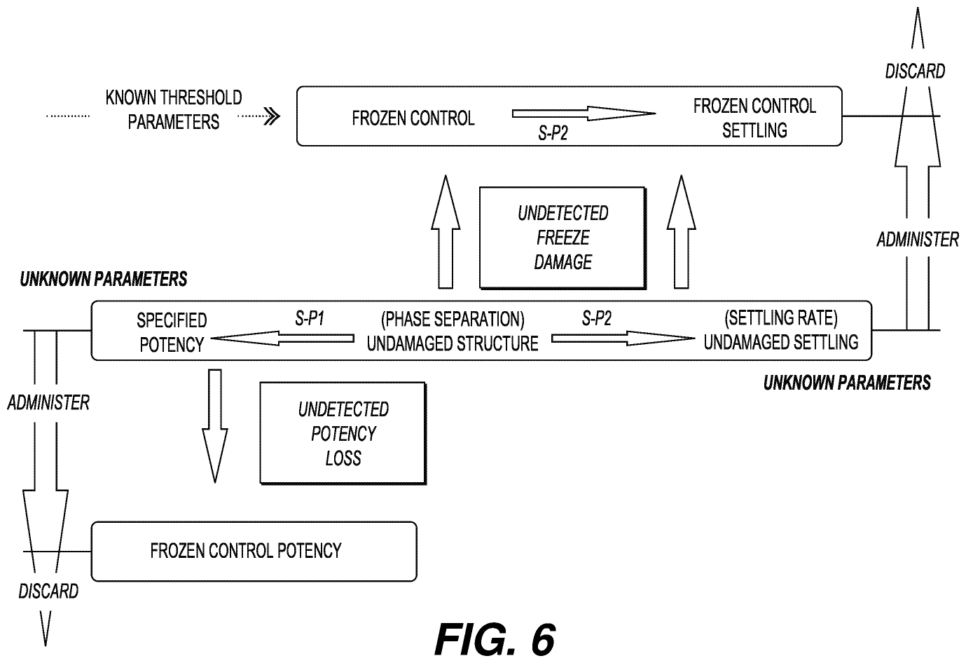

Those with skill in the art are aware that the World Heath Organization (WHO) endorses a “Shake Test” method for testing vaccines. The Shake Test and U.S. Pat. No. 9,013,699 (the '699 patent), incorporated herein by reference in entirety) make use of structure-property relations to qualitatively evaluate adsorbed vaccines for evidence of freeze-damage by applying a basic “threshold model” heuristic (see e.g., FIG. 6). Such models typically rely on some established threshold value upon which to derive a binary decision (yes/no type of decision). For example, threshold models are routinely utilized in toxicity decisions wherein exposure levels below a specific threshold are considered “safe” while those at or above the threshold value are considered “unsafe.” Threshold models are intended only to indicate whether a singular and hyper specific event of interest has reached some critical value. By design, threshold models are fundamentally unable to identify the absence of the critical event for which they test for. The Shake Test and the '699 patent seek to evaluate an unopened and suspect vial of a vaccine for evidence of freeze-damage by comparing the settling rate of the suspect vial to that of an intentionally frozen control vial drawn from the same presentation. Whereas the Shake Test relies on a human observer for this comparison, the '699 patent may utilize a single dimension sensor, i.e., a 1-D optical sensor that is merely configured to test lateral position vs. time. The '699 patent teaches if the suspect vaccine settles at the same or higher rate as the frozen control, it is determined to be freeze-damaged and discarded. Otherwise, the suspect vaccine is determined to be undamaged and is deemed safe for patient administration.

The heuristic of the '699 patent is strictly valid only under three conditions. First condition, for all vaccine presentations, the frozen control must visually display some degree of freeze-damage. Otherwise, the test may produce a false-positive result (i.e., good vaccine being discarded). Second condition, for any given vaccine presentation, the data associated with the frozen control of the vaccine must represent a lower threshold for the extent of freeze-damage possible in that presentation. For example, the material state specification must be a material state exhibiting severe freeze damage that is sufficient enough for always discarding a bad vaccine. Otherwise, the test may produce a false-negative result (i.e., bad vaccine being administered). This test has the limitation of not being able to sense freeze damage of a lesser extent that may have occurred several times. As explained above, in the HepB Vaccine type, repeated mild freeze damage can have significant adverse effects as to vaccine potency. Third condition, the measurement method used for comparing suspect and control vaccines must be applied in an equivalent manner each time by each participant of the supply chain. Otherwise, the test may produce false-positive and/or false-negative results.

Regardless of presentation, the WHO Shake Test and the '699 patent protocol dictates that the frozen control be prepared by exposing a suitable vial of vaccine to a single freeze-thaw cycle of −20° C. overnight (approximately 12 hours). This represents an “extreme exposure event” and was most likely chosen due to the fact that the freezer compartment of most available refrigeration systems is operationally limited to a temperature range between −18° C. and −22° C. Although this set of exposure parameters may produce freeze-damage in many currently produced adsorbed vaccines, use of the Shake Test continues to present a potential for false-positive results. Due to a wide variation in the intrinsic settling rates of different presentations, it is often difficult for a human observer to accurately differentiate between undamaged and freeze-damaged vaccines by visual inspection. For one presentation, a clear distinction may be made within three minutes while another may require an hour because there is no standard defined time limit for its completion. The Shake Test is increasingly likely to produce false-positive results when the intrinsic settling rate of vaccine compositions decreases.

Another difficulty with the Shake Test, is when one or two-dose presentations are being evaluated because these presentations have small volume of liquid available for visual comparison. The '699 patent sought to improve the Shake Test accuracy by replacing the human observer with an optical sensor meant to better identify differences in settling rate. This may have restricted the incidence of false-positive results to cases in which the frozen control samples were improperly prepared (i.e., not sufficiently freeze-damaged to reduce the potency of the vaccine), the potential for such human error is continually present throughout the pharmaceutical cold chain.

Problems with the second stated condition are of a far more fundamental nature. The onset of phase separation in adsorbed vaccines may begin at significantly higher temperatures than the arbitrary −20° C. value often used for frozen control preparation, e.g., −5° C. for some presentations. Furthermore, previous work by PATH on adsorbed HepB vaccines has experimentally demonstrated that the progression of phase separation does not follow a simple Heaviside step-function wherein once initiated, freeze-damage immediately advances to the extent displayed by a frozen control. While vaccine presentation may certainly mediate its rate, the progression of freeze-damage is a complex function impacted by temperature, time, and/or freeze-thaw cycles. Accordingly, a frozen control prepared in accordance with Shake Test or the '669 patent protocol does not represent a lower threshold for the extent of freeze-damage in an adsorbed adjuvant vaccine presentation. As displayed in FIG. 6, any degree of freeze-damage in a suspect vial of a vaccine which is less than that present in the control vial to which it is compared may therefore, go undetected if it settles more slowly, i.e., if it has a natural slower settling rate from a material property perspective. Accordingly, a constant risk of false-negative results exists in which freeze-damaged vaccine may be unfortunately administered to patients. As explained above, this may be a direct consequence of the underlying Threshold Model which is designed only to indicate whether an event (freeze-damage) has reached a specific threshold level and therefore, may not identify the occurrence of events below this value.

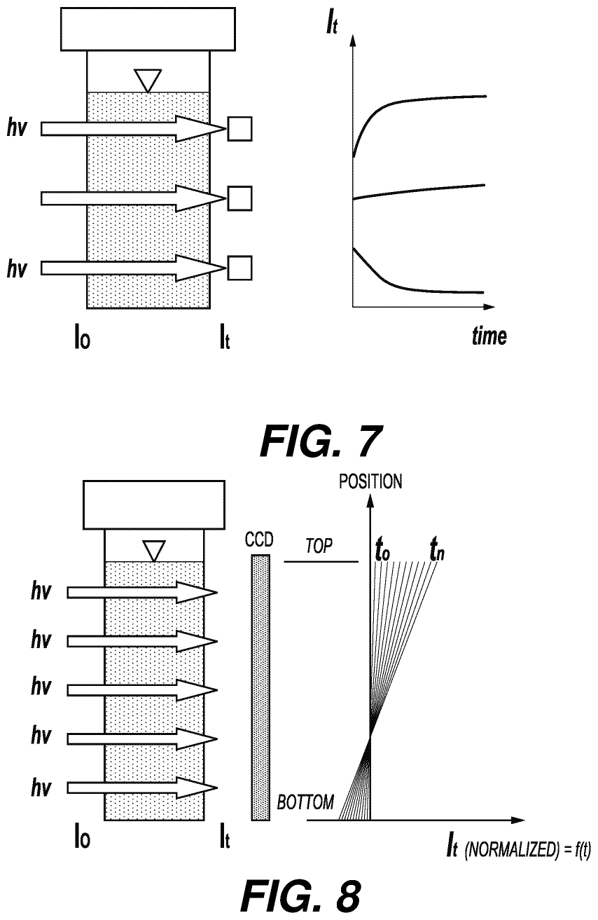

The third condition may refer to measurement equivalence, which is a general requirement for any comparison-based analysis methodology and is of critical importance in the present case. In addition to its material dependence, the optical characteristics of a liquid vaccine suspension are position dependent as well. As seen in FIG. 7 a sedimentation profile (transmitted light intensity vs. time) also displays position dependence of the detector along the direction of sedimentation. FIG. 7 illustrates sedimentation profiles produced from three hypothetical 1-D light detector positions near the top, middle, and bottom of an adsorbed vaccine liquid suspension. Because both the Shake Test and the '699 patent rely upon the arbitrary preparation of frozen controls as comparison sources within a Threshold Model heuristic, their aims are necessarily constrained to only identifying an extent of freeze-damage equal to, or greater than, that displayed by those frozen controls. Consequently, both methods are fundamentally unable to identify the absence of phase separation (freeze-damage) with any specified accuracy. Referring to FIG. 7., a sedimentation profile generated by a single, 1-D light detector will appear different when that detector's position is varied along the direction of sedimentation. In the example, sediment is progressively moving away from a detector, e.g., a photodetector, light detector, camera, lens etc. that is positioned near the top of a suspension column. Conversely, if the detector is positioned near the bottom of the suspension column, the sediment progressively moves towards the detector. Additionally, there necessarily exists some intermediate position in which equal amounts of sediment are moving towards and away from a detector simultaneously. In the absence of specific strategies to ensure that the light detector described in the '699 patent is precisely positioned in an equivalent manner, the use of the '699 patent in comparing the settling characteristics of a suspect vial of vaccine to those of any other vial will produce false-positive and/or false-negative results of an indeterminate degree. Output presented as originating from the system of the '699 patent can only be generated by a single, 1-D light detector positioned at a specific location along the direction of sedimentation flow. This design positioning becomes increasingly problematic as the volume of liquid being evaluated is reduced to a small ml of about 0.5 ml (i.e., for single-dose vaccine presentations).

### Example Data Generation Techniques

The techniques disclosed herein allow for quantitative AND qualitative identification of both the absence and incidence of phase separation (freeze-damage) to any extent, with no false-positive or false-negative results. The techniques are quantitative in that a number, e.g., a percentage, may be assigned to the incidence of phase separation for any number of samples. The techniques are qualitative in that a number, e.g., a percentage, may be assigned to the incidence of phase separation that is indicative of the extent/severity of phase separation which may be correlated to a corresponding reduction in potency of an SDPP, e.g., a potency reduction in a vaccine.

The techniques disclosed are rapid, may require no special preparation of frozen controls, and are applicable for use at all points along the pharmaceutical cold chain. As explained above in conjunction with FIG. 2, this may be accomplished through the application of a Baseline Model heuristic. This Baseline Model heuristic may be dependent upon two primary conditions. First condition, the sample from which the Baseline Dataset and Specified Representation were derived must accurately display both, the specified potency (or potency of interest) and specified material state (or state of interest). Otherwise, the test may produce false-negative results. Second condition, the measurement methods utilized to produce the Specified Representation must be applied to the specimen vaccine (to produce the Specimen Representation) in an equivalent manner. Otherwise, the test may produce false-positive and/or false-negative results.

Considering the example of an adsorbed vaccine as previously described, the first condition may be satisfied by verifying the specified potency and material state of representative sample vaccines using assays and phase-contrast microscopy, respectively. Within a vaccine production context, both the optical analysis and this type of As-Specified verification may be performed immediately following manufacture (e.g., a manufacture potency) or just prior to product release (e.g., a release potency). This high level of certainty is not possible for the field-prepared frozen controls upon which the Shake Test and the '699 patent rely. The second condition may be satisfied by evaluating a sufficiently large area of each vaccine vial or container such that measurement windows overlap, and thus equivalence is assured.

Different from the '699 patent example disclosed, material state testing machines may utilize a 2-D optical sensor such as a line-camera or CCD array (i.e., lateral position, and axial position vs. time). For example, when a 2-D optical sensor is applied to a vial of adsorbed adjuvant vaccine along the direction of sedimentation, it may produce an assembly of different sedimentation profiles. As seen in FIG. 8, this assembly may be converted into a 3-D optical profile (e.g., a Photonic Profile). As used herein, the term “Photonic Profile” may refer to the temporal progression of normalized transmitted light intensity versus vaccine vial position. FIG. 8 illustrates application of a 2-D optical sensor (CCD) to the evaluation of an unopened vial of adjuvanted vaccine that produces a 3-D Photonic Profile which displays the temporal progression (t₀-tₙ) of transmitted light versus vial position (top to bottom).

As previously discussed, a single sedimentation profile is not unique to any specific vial of adsorbed vaccine due to its position dependence. This is also true of any individual sedimentation profile from within the assembly of profiles produced by a 2-D sensor. However, the specific assembly of those profiles is unique because it presents a high-resolution record of the distinct manner in which the entire vaccine dispersion changes as a function of time. The resultant Photonic Profile presented in FIG. 8 may constitute a unique “data-representation” for a given vial of vaccine such that virtually any structural change in composition (particle size and/or size distribution) due to freeze-damage may be readily detected by analyzing the Photonic Profile for differences in material state vs. the control sample/known good sample.

### Testing Pentavalent Example

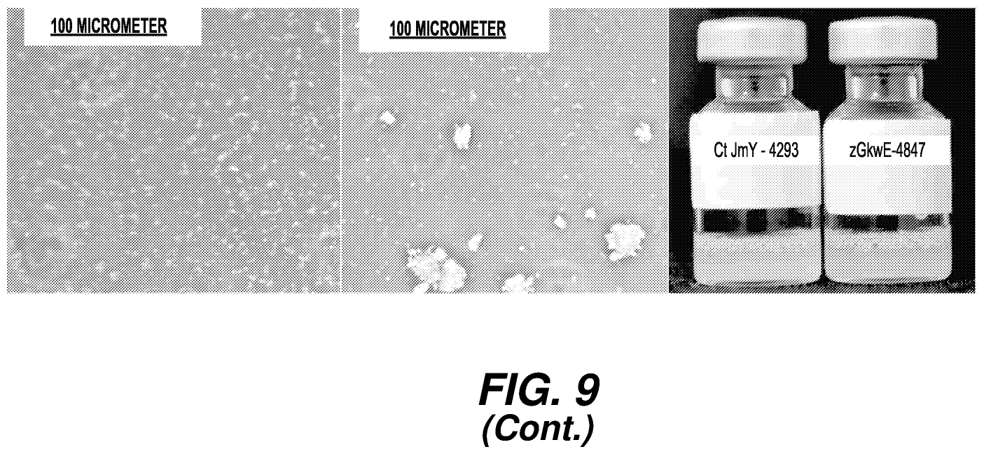

This degree of precision is largely retained for specific vaccine presentations, as is displayed in the experimental example of FIG. 9. An 850 nm light-emitting diode (LED) coupled to an optical sensor and high-speed data acquisition card was utilized to evaluate Pentavalent vaccines from Panacea Biotec in a single-dose presentation. The system of FIG. 9 is meant to emulate the 2-D optical sensor and associated 3-D output data of FIG. 8 above. FIG. 9 illustrates Photonic Profiles of single-dose presentations of Pentavalent Vaccine in As-Specified (upper left) and Freeze-Damaged (upper right) conditions. Phase-contrast microscopy of As-Specified (lower left) and Freeze-Damaged (lower middle) vaccines are presented as well. The lower right image displays these vaccines one hour following homogenous dispersal. Shown, are As-Specified (CtJmY) and Freeze-Damaged (zGkwE) examples.

In this example, the Pentavalent is intended to protect children from five of the most serious diseases (Diphtheria, Pertussis, Tetanus, Hepatitis B, and Haemophilus influenzae type b). This particular Pentavalent is among the most sensitive to freeze-damage. The vial labeled as “CtJmY” was continuously maintained at 5° C. and represents vaccine in “As-Specified” condition. This was verified by phase-contrast microscopy which displays a relatively monodisperse size distribution of fine antigen-adjuvant particles (lower left). The vial labeled as “zGkwE” was exposed to a single freeze-thaw cycle of 24 hours at −10° C. and represents a specimen of vaccine for which freeze-damage is suspected. As indicated by phase-contrast microscopy, this low temperature treatment resulted in phase separation (e.g., freeze-damage) which produced large adjuvant agglomerates (lower middle).

The lower right image of these vaccines was taken one hour following homogenous dispersal per the WHO Shake Test and the '699 patent protocol. Even when viewed against a black background with optimal illumination, no visible difference in sedimentation characteristics is observed. This remained the case as both vaccines continued to settle completely over the next hour. It is important to note that if the Freeze-Damaged vaccine “zGksE” were utilized as a Frozen-Control to evaluate the As-Specified vaccine “CtJmY,” application of the Shake Test would produce a false-positive result. Due to their specific architecture, Pentavalent vaccines typically display a relatively slow intrinsic settling rate and thus, this example demonstrates a distinct advantage of the present technology in freeze-damage evaluation of SDPP.

In at least some highly efficient embodiments, Photonic Profiles may be presented for each vaccine and comprise just five minutes of optical data (upper left and right). The As-Specified profile (e.g., Specified Representation) may be characteristic of a relatively monodisperse size distribution of weakly interacting particles undergoing steady sedimentation. Note the cross-over from increasing to decreasing signal intensity near the suspension column center at ˜4.5 along the x-axis. In contrast, the Freeze-Damaged profile (e.g., the Specimen Representation) is characteristic of a polydisperse distribution of particles settling at a significantly higher rate. Simple calculations involving a summation of the intensity differentials indicates a 3.6 times difference for the “freeze-damaged” specimen vaccine relative to the “As-Specified” baseline vaccine. Therefore, this example embodiment shows a vaccine material state change (e.g., freeze-damage) has been accurately detected by comparing a Specimen Representation to its Specified Representation according to disclosed techniques.

### Data Evaluation Embodiments

Greater sophistication in the evaluation of these datasets may be accomplished using machine learning. This is particularly useful due to the inherent complexity of both the vaccine materials being evaluated and the associated measurement data generated. Again, taking the example of an adsorbed adjuvant vaccine as displayed in FIG. 9 above, each vial of material is not exactly the same as others taken from a given batch and each batch itself, is often substantially unique. This stems from the synthesis methods used for vaccine production which typically yield a somewhat non-homogeneous colloidal suspension. Accordingly, the settling characteristics of individual vials taken from the same batch may display slightly different properties. Furthermore, the optical data produced from the same vial may differ slightly from measurement to measurement due to differences between shaking and settling events. Evaluation of measurement data under these circumstances may be aided by an iterative categorization heuristic as commonly used in machine learning methods.

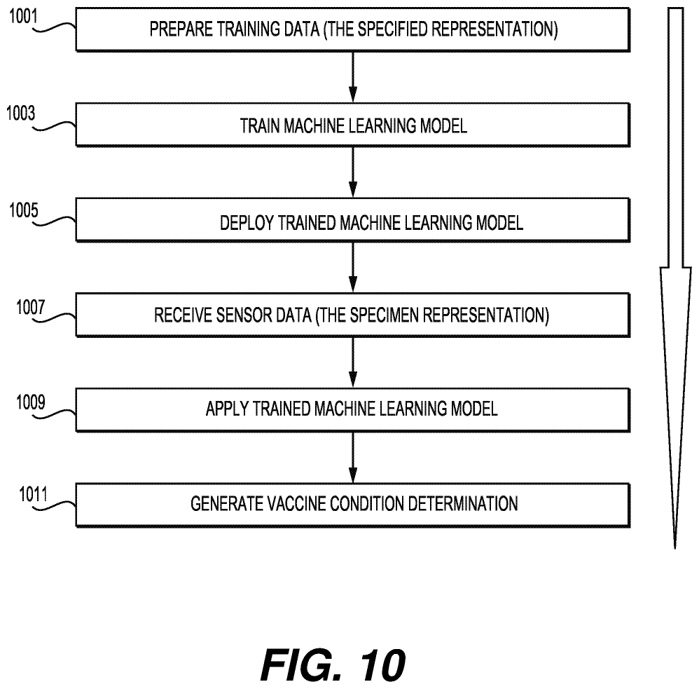

FIG. 10 is a flow diagram illustrating an embodiment of a process for training and applying a machine learning model to evaluate Specimen Representation data. In some embodiments, the process of FIG. 10 may be used to determine if a vaccine specimen is characterized by the specified material state as defined by an applicable Specified Representation. The sensor data used for training and/or the application of the trained machine learning model may derive from, and correlate to, measurements used to produce the Specified Representation.

At step 1001, training data may be prepared. In some embodiments, sensor data taken from vaccines with known material properties is used to create a training data set. The data may include electrical, chemical, acoustic, optical, etc. At least one optical example may be similar to a machine capable of generating photonic profiles and/or sensor data like FIG. 8. from one or more sensors acting on such vaccines. The prepared training data may include data for training, validation, and testing. For example, vaccine specimens that have been intentionally freeze-damaged may be included to validate the model's ability to identify such material conditions. In some embodiments, the format of the data is compatible with a machine learning model used on a deployed deep learning application.

At step 1003, a machine learning model may be trained. For example, a machine learning model may be trained using the data prepared at 1001. In some embodiments, the model may be a neural network such as a convolutional neural network. In various embodiments, the model includes multiple intermediate layers. In some embodiments, the neural network may include multiple layers including multiple convolution and pooling layers. In some embodiments, the training model may be validated using a validation data set created from the vaccine samples with known properties (i.e., undamaged and/or freeze-damaged). In some embodiments, the machine learning model may be trained to predict whether a vaccine specimen's material state has changed to an extent that it is no longer suitable for human or animal administration.

At step 1005, the trained machine learning model may be deployed. For example, the trained machine learning model may be installed on a vaccine evaluation device. In some embodiments, this may be accomplished by an over-the-air firmware update transmitted using a wireless network, such as a WiFi or cellular network. In some embodiments, the newly trained machine learning model may be located on a server with restricted access. In additional embodiments, this server may be part of a Blockchain ledger system.

At step 1007, sensor data comprising the Specimen Representation may be received. In some embodiments, these sensors are equivalent to those used to produce the training data prepared at step 1001. At step 1009, the trained machine learning model may be applied. For example, the machine learning model trained at 1003 may be applied to sensor data received at 1007. In various embodiments, by applying the trained machine learning model, the degree to which vaccines comply with their material specifications, as defined by the Specified Representation, may be determined. At step 1011, a determination of vaccine condition may be generated. For example, evaluation of the Photonic Profile (top right) displayed in FIG. 9 above would indicate that the specimen labeled zGkwE may be freeze-damaged as compared to the vaccine specimen labeled CtJmY.

### Data-Communication Embodiments

While the system and method described thus far may be capable of identifying even the most subtle extent of potency-correlated vaccine state change, its accuracy may be compromised by unintentional or intentional data corruption. In particular, communicating the Specified Representation, upon which vaccine assessment ultimately relies, must be secured. Furthermore, there can be no assurance that an accurate vaccine assessment will be properly recorded and acted upon by program participants (i.e., vaccine developers, manufacturers, freight shippers, government customs officers, last-mile couriers, medical storage facilities, healthcare workers, etc . . . ). These dual issues of data integrity and assessment auditability may be addressed through the application of data encryption and/or Blockchain ledger technologies. In various implementations, “the Blockchain” may describe a public and immutable ledger consisting of a plurality of data blocks wherein successive entries are recorded and immune from changes/unauthorized tampering of the data blocks by using cryptography. Accordingly, a blockchain ledger may be an effective network for communicating the Specified Representation and outcomes of Specimen Evaluations, to participants across the supply chain network. A blockchain ledger and network provides entities in the vaccine distribution system with a non-falsifiable chain of custody for every vaccine specimen in the system. This provides complete auditability for assurance of suitable potency, at any time, including that of administration. Many different blockchain architectures are known and available, varying according to whether the blockchain may be localized or distributed, whether the method of transaction verification may be proof-of-work, proof-of-stake, or some other method, whether the block chain may be private or public, whether or not tokens are used to track events, and so on. Accordingly, those skilled in the art will be able to select from a wide range of these configurations when implementing a blockchain ledger system to communicate vaccine state data such as the Baseline Dataset, the included Specified Representation, the Specimen Representation, and its evaluation results, within the system and method described herein.

While several data security/encryption schemas are applicable within such a Blockchain system, Homomorphic encryption may be of particular utility in the present case. Homomorphic encryption may be a form of encryption that permits users to perform computations on encrypted data without first decrypting it. These resulting computations are left in an encrypted form which, when decrypted, result in an identical output to that produced had the operations been performed on the unencrypted data. Homomorphic encryption can be used for privacy-preserving of outsourced storage and computation thus, allowing data to be encrypted and out-sourced to commercial cloud environments for processing, all while encrypted. Further, the computations applied to such encrypted data may be carried out with a trained machine learning model such as that previously described in conjunction with FIG. 10. This method of encryption allows the Specimen Representation to be compared against the Specified Representation while both are encrypted. As a result, the Specified Representation (and/or the associated trained machine learning model) can be encrypted prior to transmission, ensuring that no participant or stakeholder in the system knows what the qualities of the Specified Representation are, so no participant or stakeholder may be able to duplicate the Specified Representation or otherwise able to use the Specified Representation to generate false specimen measurement results.

Another important aspect of homomorphic encryption may be that it allows for the comparison of encrypted data sets in order to evaluate their similarity without requiring that one data set be strictly contained within another. This may be of particular importance for assessing vaccines and other state-dependent pharmaceuticals because their potency-correlated states (i.e., charge state, structure, chemical or antigen affinity, electromagnetic, etc.) are known to change in response to a variety of conditions (i.e., improper manufacturing and/or handling). The system and method described herein are explicitly developed to detect such material state changes. This differs fundamentally from object data comparison methods explicitly designed for counterfeit detection, such as those described in U.S. Pat. No. 10,193,695, (the '695 patent) wherein deviation from an otherwise “unclonable function” may be evaluated. Such functions are to be immutable with any aging effects explicitly known and accounted for within the cryptographic hash system and methods. Application of such strict cryptographic hash comparisons in the present case of vaccine assessment, would produce the appearance of a counterfeit every time because the material states upon which such assessments are focused routinely change as previously discussed (i.e., a given vaccine can be authentic, yet non-viable).

In fact, a vaccine may be authentic and viable, yet still display some acceptable material state change. Homomorphic encryption allows the Specified Representation or the inference engine corresponding to its characteristics to be used for evaluating the Specimen Representation via comparison and/or machine learning classification. In some embodiments, this produces an evaluation result and a directive according to the manufacturer specifications, rather than just a variance between two encrypted hash values, as may be the case for counterfeit detection per the '695 patent.

In some embodiments, data containing the Specified Representation of a vaccine presentation may be added to a Blockchain system. For example, the Blockchain system may be created specifically for storing and tracking information about vaccine state. In another embodiment, this data may be encrypted using asymmetric homomorphic encryption using the private key of the manufacturer and digitally tagged with a specification identifier for the corresponding vaccine presentation to which that Specified Representation applies. For example, the Specified Representation data may be of the form previously displayed in FIG. 9 (top-left). At some later time, in various embodiments, a vaccine specimen may be created and labeled with both a specification identifier corresponding to the applicable Specified Representation and a unique specimen identifier corresponding uniquely to that physical vaccine specimen.

At some later time after creation, the vaccine specimen may be selected for compliance assessment. In various embodiments, prior entries in the blockchain corresponding to that vaccine specimen are then obtained using the unique specimen identifier of that vaccine specimen. If the prior entries indicate that the vaccine specimen has previously been flagged for wastage or return, the user may be informed of these results and directed accordingly. If there is no prior entry, or all prior entries indicate a history of compliance, a data representation of the specimen may be created according to the previously discussed methods. This Specimen Representation may be encrypted with homomorphic encryption using the private key of the user performing the assessment. In some embodiments, encrypted data containing the most recent Specified Representation that corresponds to the identifying information of the vaccine specimen may be then obtained from the blockchain. A comparison may be then performed between these two data sets wherein their similarity may be evaluated and classified with a compliance status according to the Specified Representation. A data set containing the specimen identifying information and representation, compliance assessment, and ancillary information, may be added to the blockchain. In further embodiments, the user may be informed of the compliance status of the vaccine or other state-dependent pharmaceutical and directed accordingly.

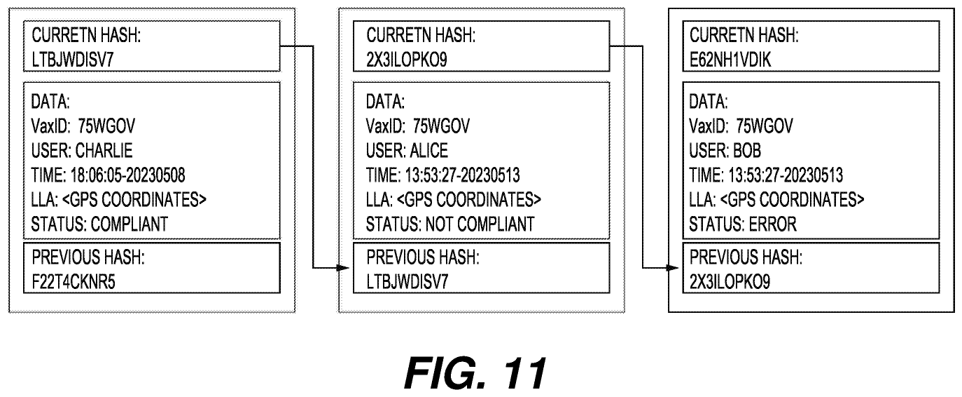

The moment that any specimen may be evaluated to have an excursion from the specified state, indicating that it should be spoiled and discarded, every participant in the pharmaceutical product distribution system will know that that specimen should no longer continue through the system towards administration. FIG. 11 illustrates an example of the blockchain implementation of the system and method described herein. It illustrates how the record of hashes ensures that no prior record can be altered after it has been authenticated and entered into the blockchain. Suppose that a vaccine is in the system and method described herein. Charlie, a participant in the cold chain does a spot check on this vaccine and finds that it is compliant. Later, Alice tests that same specimen, but the result is not in compliance with the specification. Instead of following the evaluation result directive to spoil and discard the tested specimen, Alice instead chooses to send the specimen forward to the next location in the pharmaceutical distribution network. The result of this test will already be recorded in the blockchain ledger for the pharmaceutical distribution network. Later, when Bob gets the specimen and tests it in a specimen measurement device, the evaluation result will immediately flag the specimen and implicate Alice for having failed to abide by a previously generated evaluation result directive. Bob now has evidence that the SDPP material state change happened while the specimen was in Alice's custody, not his, and he can seek a remedy from Alice for having been sent a specimen that was known to be outside of its specifications.

Because of the immutability of such a blockchain ledger, there may be no way that the entries could have been changed after the fact. The evaluation result records would not only implicate Alice for knowingly sending mishandled pharmaceutical product closer to administration, but also potentially Charlie, depending on who had custody of the vaccine vial between those measurements. In this circumstance, only a single specimen was flagged for spoilage, but examination of the evaluation result by the manufacturer or other authorities could result in a larger grouping-such as an entire shipment or batch-being flagged for spoilage and discarded. Because these changes would be recorded on the blockchain and made available remotely, they would be instantly available to all stakeholders in the blockchain ledger being used to manage the pharmaceutical product distribution system.

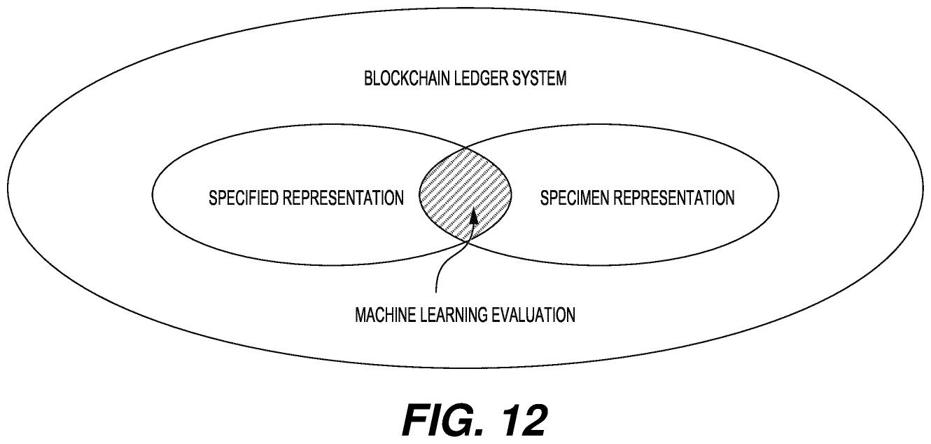

An embodiment for the entire system and method comprising specified and specimen vaccine state data representations, their evaluation using machine learning, and the communication of all data and associated evaluations within a blockchain ledger system, may be presented in the Venn Diagram of FIG. 12.

FIG. 12 illustrates a Venn Diagram presenting an embodiment for a complete system and method that incorporates vaccine data generation, machine learning evaluation, and blockchain encryption/communication. With the inclusion of methods to transmit or convey the Baseline Dataset and/or the Specified Representation, a functional system may be envisioned which accurately applies the Baseline Model Heuristic to ensure that state-dependent pharmaceuticals such as vaccines (or vaccine components) have retained their specified properties prior to patient administration. Such a system may be comprised of some, or all of the following core components and their various embodiments.

### Measurement Data:

Data comprising the Specified Representation and/or Specimen Representation may be derived from one or more sensors that evaluate measurable properties directly correlated to distinct potency-correlated vaccine states. For example, in some embodiments, optical sensors capture light which has been transmitted and/or scattered through a transparent container of liquid vaccine. While this disclosure has focused on vaccines contained within their final packaging (vial, ampule, or single-dose syringe), similar optical analyses may be performed on a volume of liquid vaccine drawn directly from a batch, at any point during the manufacturing process. Accordingly, some embodiments may refer to datasets and subsequent evaluations on unpackaged liquid vaccines. Such capabilities may substantially improve the quality control process thus, saving vaccine manufacturers from the significant monetary expense associated with releasing damaged and/or out-of-spec products. One embodiment utilizes optical data which originates from a 2-D sensor such as a line-camera or CCD array. As previously discussed, this may be converted into a 3-D optical profile (Photonic Profile) which serves as a unique data-representation for a liquid vaccine suspension due to its high degree of structure-property correlation. Additional embodiments may utilize optical data originating from one or more 1-D, point detectors chosen to detect specific electromagnetic wavelengths which can indicate a shift from Rayleigh to Mie scattering regimes. As previously discussed, a principal advantage of using a 2-D optical sensor may be its ability to offer measurement equivalence. In some embodiments, the number and position of such sensors (2-D and/or 1-D) may be varied to cover a portion (or substantially all) of the liquid vaccine suspension.

### Baseline Dataset:

The Baseline Dataset comprises a comprehensive set of data which describes the applicable vaccine category, vaccine condition, measurement parameters, and Specified Representation data produced. Each of these elements may be important to ensure that measurement equivalence and thus, evaluation accuracy may be attained.

### Vaccine Category:

The vaccine category refers to the range (broad, narrow, or specific) of vaccine presentations to which the Specified Representation may be applicable. In some embodiments, a broad category may be “all adsorbed adjuvant vaccine presentations.” For example, this may be based upon one or more characteristics of the Specified Representation data that may be common across all presentations such that any specimen (regardless of presentation) may be evaluated by noting the absence or incidence of this characteristic(s). In some embodiments, this information may be directly programmed into a single device used to evaluate all adsorbed adjuvant vaccines without the need for baseline dataset or Specified Representation communication/transmission. Another embodiment considers the vaccine category to reflect a narrow vaccine presentation which comprises a distinct combination of (vaccine type, vaccine manufacturer, batch number, and doses per vial). In this case, baseline data and the associated Specified Representation will reflect the characteristics of a specific batch of vaccine. In another embodiment, the category may be further narrowed to focus on a single vial of vaccine from that presentation. This entails gathering sensor data from a specific vial of vaccine and then later using that data for subsequent evaluation of that same, specific vial of vaccine. This extreme degree of material state monitoring may be of particular benefit for vaccine development efforts wherein the performance of various vaccine candidates often carries significant financial and product approval impacts. A further benefit of this embodiment may relate to monitoring individual doses of SDPPs that are tailor-made for a specific individual (e.g., personalized cancer vaccines which are unique to a patient's tumors).

### Vaccine Condition:

The vaccine condition refers to the specified potency and specified material state being reflected in the Baseline Dataset and Specified Representation. At this time, there is no scientific consensus regarding a safe level of freeze-damage in vaccines. Accordingly, one embodiment utilizes a vaccine condition that corresponds to “no freeze-damage.” However, in the event that an allowable degree of freeze-damage (or other material state change) is to be accommodated, some embodiments may utilize vaccines which reflect that allowance.

### Measurement Parameters:

The measurement parameters refer to device specifications and settings associated with data generated from the vaccine sample space(s) which comprise the Baseline Dataset and Specified Representation. This information may be needed to ensure that measurements of specimen vaccines to which the Specified Representation may be applicable, are performed in an equivalent manner. The baseline data produced describes the Specified Representation data originating from sensors operating on the category sample vials (or other containers) of vaccine. In one embodiment, this sensor data takes the form of a 3-D optical profile (Photonic Profile) as previously presented in FIGS. 8 and 9. In additional embodiments, this data may be the raw data from which the Photonic Profile was derived or a calculated scalar value derived from that raw data.

### Baseline Dataset Transmission System:

The primary reason that both the Shake Test and the '699 patent rely on known “frozen” controls for vaccine evaluation may be because known “Unfrozen” controls do not exist in the field environment. Prior to intentional freezing and/or testing, every vial of vaccine within a given shipment ultimately presents an unknown condition. One advantage of the disclosed technology may be its reliance on a Specified Representation generated from known “Unfrozen” controls in a laboratory environment. However, that data must be transmitted or conveyed in some manner such that it may be available for use in specimen evaluation. In one embodiment, this may be accomplished by uploading the electronic dataset files to a server which may be accessible through the internet. In other embodiments, this electronic data may be saved to storage media such as a removable flash memory card or in non-removable media within a testing device itself. Additional embodiments may rely on encoding one or more processors or application specific integrated circuits (ASICs) within a vaccine testing device. This approach may be utilized for the previously described broad vaccine category case. As previously discussed, various embodiments may utilize a calculated scalar value rather than a data file. This dataset may take the form of a value, data file, data structure, locator, or data tracker which links to a web site to obtain such information. It may be printed directly onto paper inserts and/or packaging (including the vaccine vial label) in the form of one or more QR codes or bar codes. The Baseline Dataset may take the form of a radio frequency identification (RFID) tag or other electronic media that contains said data located on, or otherwise physically associated with, a vaccine vial or collection thereof. Additional embodiments may utilize a Blockchain ledger system as previously described.

### Specimen Dataset:

Similar to the Baseline Dataset, the Specimen Dataset comprises a comprehensive set of data which describes the vaccine category, measurement parameters, and specimen data produced. A description of the vaccine category may be needed to ensure that specimen data may be evaluated with respect to the correct baseline data and measurement parameters are required to ensure that measurement equivalence may be retained. The specimen data produced describes the data originating from sensors operating on the specimen vials of vaccine selected for evaluation. In one embodiment, this data takes the form of a 3-D optical profile (Photonic Profile) as previously presented in FIGS. 8 and 9. In additional embodiments, this data may be the raw data from which the Photonic Profile was derived or calculated scalar values derived from that raw data.

Comparison Algorithm: Accurate evaluation relies on comparing specimen data (the Specimen Representation) to its applicable baseline data (the Specified Representation). As previously described for the experimental example of FIG. 9, calculations involving a summation of the intensity differentials may be used for cases in which significant freeze-damage may be present. However, cases of relatively minor freeze-damage may require a greater degree of sophistication in the evaluation of associated data. In anticipation of a desire to detect even the smallest amounts of freeze-damage, one embodiment utilizes machine learning methods such as Convolutional Neural Networks (CNN) to evaluate specimen data with respect to its applicable baseline data. In other embodiments, the CNN or other neural network may be trained with supervision via back-propagation to classify the vaccine as viable (acceptable material state change) and/or as non-viable (unacceptable material state change). In further embodiments, the CNN or other neural network may be trained to output a probability that the vaccine is viable and/or non-viable. Additional embodiments involve training a machine learning model to recognize and categorize specimen vaccines within a non-viable sample range. For example, the manufacturer may determine the most likely ways that a vaccine state would fail or change to a point that its potency falls below a critical threshold value. In some embodiments, a specific profile and/or inference engine may be deployed to detect such cases. For example, data corresponding to viable, barely viable, barely non-viable, and non-viable may be used to train such a machine learning model or inference engine. In some embodiments, the inference engine uses these as classifiers in a majority vote process to determine vaccine viability status. In further embodiments, each of these may be run in parallel to provide a fast result or other benefits.

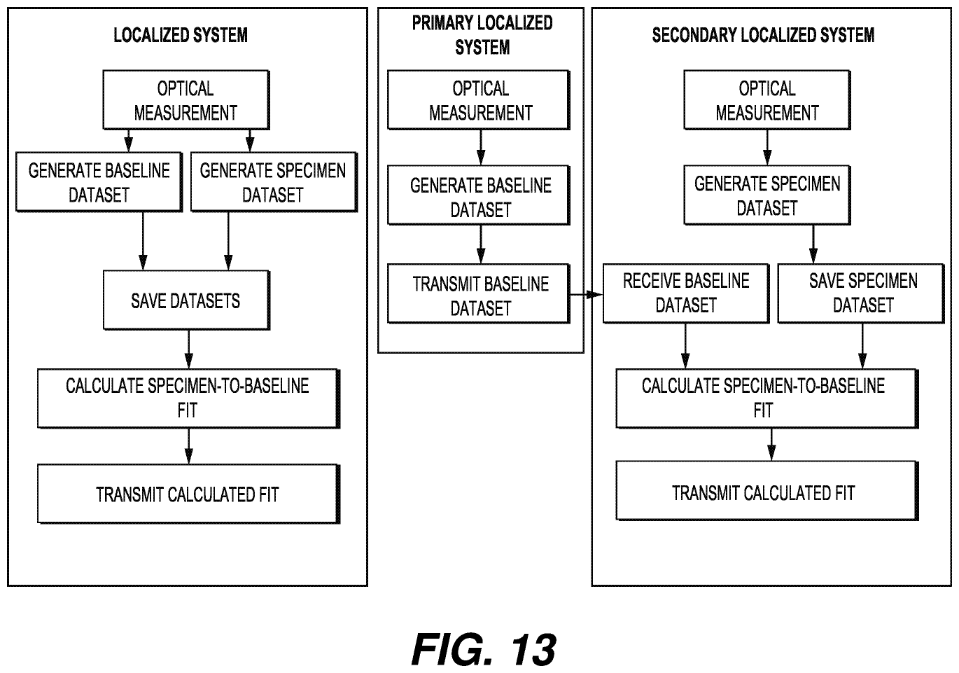

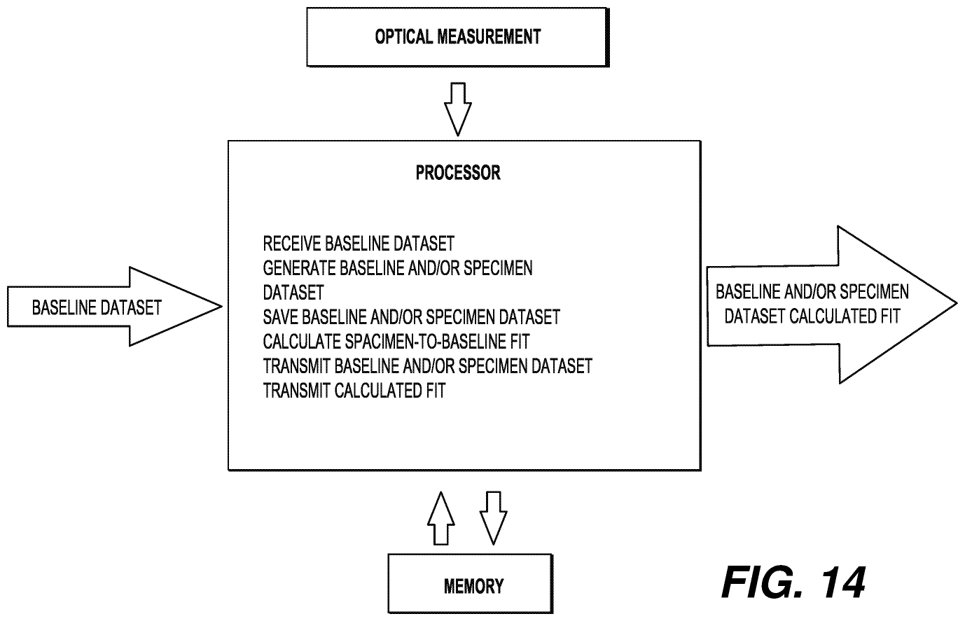

Comprised of these components, we may envision two categories of such a functional system, one localized and the other distributed. A localized system describes the core technologies (hardware, software, and method), through which the Baseline Model Heuristic may be applied, within a local device to evaluate vaccines for evidence of material state change. A single, localized system may be applied within a vaccine development program to ensure the integrity of costly clinical trials or incorporated into a vaccine manufacturing process to enhance final product quality control (FIG. 13 left). FIG. 13 illustrates an embodiment of Localized (left) and Distributed (right) functional systems for applying the Baseline Model Heuristic to vaccine evaluation. Working in concert with additional elements, a distributed system describes the process by which a series of such localized systems (Primary and Secondary) may broadly apply the Baseline Model Heuristic throughout an entire pharmaceutical cold chain segment to determine whether vaccines have retained their specified properties prior to patient administration (FIG. 13 right). Such a conception regards each element of this process (vaccine specimens, measurement systems, datasets produced, methods of their transmission, and comparison algorithms) as components of the distributed system. The example embodiment of FIG. 13 applies optical analysis to adsorbed vaccines as previously discussed. In order to be of use in either isolation or in series, such a localized system must be capable of performing five distinct tasks with respect to Baseline and/or Specimen Datasets (Generate, Save, Transmit, Receive, and Calculate). The ability of a localized system to perform in these dual roles may be reflected in both the verbal description below and in FIG. 14 which follows.

FIG. 14 is an example flow chart illustrating an embodiment of a localized system configured to perform various operations in accordance with the disclosure herein. The system of FIG. 14 may include a controller and/or processor having at least one memory cell containing computer readable executable instructions stored thereon. The processor may be configured to execute the computer readable instructions to perform an operation. In at least one operation, the processor may be configured to (1) receive a Baseline Dataset comprising a plurality of sensor measurements (e.g., the Specified Representation) of a vaccine and/or vaccine component) category which has been verified to display a specified potency (or potency of interest) and a specified material state (or state of interest). Next, at step (2) the processor may be configured to generate a Baseline and/or Specimen Dataset comprising sensor measurements (the Specimen Representation) of a specimen of the vaccine (or vaccine component) category using sensor data from at least one sensor operating on the specimen. Next, at step (3) the processor may be configured to save the generated Baseline and/or Specimen Dataset for subsequent transmission and/or analysis. Next, at step (4) the processor may be configured to utilize a machine learning model to calculate a Specimen-to-Baseline Fit which comprises an evaluation of the generated and/or saved Specimen Dataset with respect to the received and/or generated Baseline Dataset. Next, at step (5) the processor may be configured to transmit the generated Baseline and/or Specimen Dataset and calculated Specimen-to-Baseline Fit. It shall be understood that the disclosed system may include at least one memory cell in communication with and/or coupled to the processor. In various embodiments, the memory cell may be custom configurable and be able to be updated automatically by a software patch or firmware update. The at least one memory cell may be and configured to store data and provide the processor with instructions.

In an alternate embodiment, FIG. 14 may be conceptualized as a computer program product, e.g., a software product that utilized the computer hardware of the processor and memory. For example, a computer program product embodied in a non-transitory computer readable storage medium and comprising computer instructions that perform an operation. In at least one operation, the computer program product may be configured to (1) receive a Baseline Dataset comprising a plurality of sensor measurements (e.g., the Specified Representation) of a vaccine and/or vaccine component) category which has been verified to display a specified potency (or potency of interest) and a specified material state (or state of interest). Next, at step (2) the computer program product may be configured to generate a Baseline and/or Specimen Dataset comprising sensor measurements (the Specimen Representation) of a specimen of the vaccine (or vaccine component) category using sensor data from at least one sensor operating on the specimen. Next, at step (3) the computer program product may be configured to save the generated Baseline and/or Specimen Dataset for subsequent transmission and/or analysis. Next, at step (4) the computer program product may be configured to utilize a machine learning model to calculate a Specimen-to-Baseline Fit which comprises an evaluation of the generated and/or saved Specimen Dataset with respect to the received and/or generated Baseline Dataset. Next, at step (5) the computer program product may be configured to transmit the generated Baseline and/or Specimen Dataset and calculated Specimen-to-Baseline Fit.

In an alternate embodiment, FIG. 14 may be conceptualized as a method that may be performed by any suitable means in accordance with the disclosure herein. In a first step, the method may include (1) receiving a Baseline Dataset comprising a plurality of sensor measurements (e.g., the Specified Representation) of a vaccine and/or vaccine component) category which has been verified to display a specified potency (or potency of interest) and a specified material state (or state of interest). Next, at step (2) the method may include generating a Baseline and/or Specimen Dataset comprising sensor measurements (the Specimen Representation) of a specimen of the vaccine (or vaccine component) category using sensor data from at least one sensor operating on the specimen. Next, at step (3) the method may include saving the generated Baseline and/or Specimen Dataset for subsequent transmission and/or analysis. Next, at step (4) the method may include utilizing a machine learning model to calculate a Specimen-to-Baseline Fit which comprises an evaluation of the generated and/or saved Specimen Dataset with respect to the received and/or generated Baseline Dataset. Next, at step (5) the method may include transmitting the generated Baseline and/or Specimen Dataset and calculated Specimen-to-Baseline Fit.

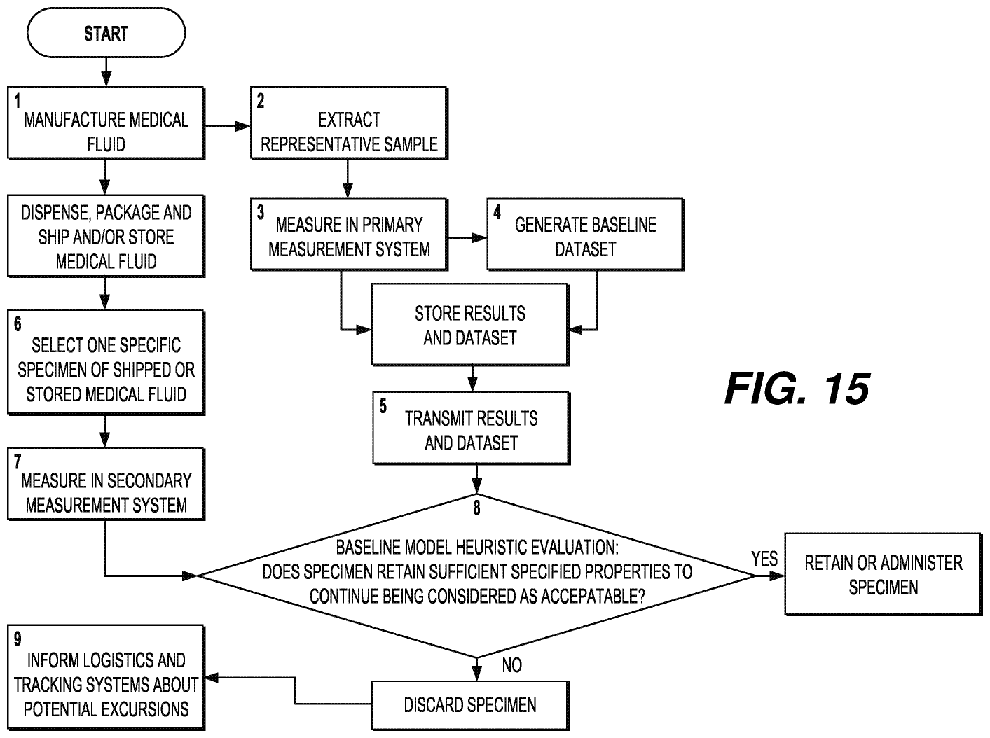

FIG. 15 is an example flow chart illustrating how a distributed system may operate. The principles of FIG. 15 may be applied in view of the various system and method embodiments described hereinabove. With reference to FIG. 15, a distributed system configured to perform a variety of steps utilizing a variety of physical hardware and physical SDPP samples is disclosed. The distributed system may include (1) A Medical Fluid, such as an SDPP for example. In various embodiments, this medical fluid may be a vaccine, or any other fluid used for human and/or animal health applications, wherein the potency or efficacy of the medical fluid is dependent upon the material state of the medically active components, and this state can be evaluated according to some directly measurable property such as (but not limited to) the optical characteristics of the medical fluid. The distributed system may include (2) A Representative Sample of the Medical Fluid. In various embodiments, the sample is selected to accurately represent the specified potency (or potency of interest) and specified state (or state of interest) of the Medical Fluid. The distributed system may include (3) A Primary Measurement System. In various embodiments, this primary measurement system observes, quantifies, and records the directly measurable property (or properties) of the representative sample of medical fluid which is (are) directly correlated to the specified potency (or potency of interest) and specified state (or state of interest), for example. Resulting measurements are converted into specified measured properties following independent evaluation of the representative sample's potency and/or material state, for example. The distributed system may include (4) A Baseline Dataset. In various embodiments, the dataset may be a record comprising the specified measured properties (or properties of interest) to which the results of measurements taken from any specific specimen of medical fluid, using a Secondary measurement system, are to be compared. The distributed system may include (5) a Baseline Dataset Transmission System. In various embodiments, this component describes the system used to store and transmit the Baseline Dataset, such that these records are available for comparison against the results of measurements on a specific specimen of medical fluid, obtained by the secondary measurement system.

The distributed system may include (6) A Specific Specimen of Medical Fluid. In various embodiments, this component describes the specific specimen of medical fluid, along with its container and packaging, wherein a determination regarding the retention of specified properties is desired. For example, this might be a specific vial of vaccine that has been selected for patient administration. The distributed system may include (7) a Secondary Measurement System. In various embodiments, the secondary measurement system may operate on the same physical principles as the primary measurement system, and is designed to observe, quantify, and record the directly measurable property (or properties) of the specific specimen of medical fluid. The distributed system may include (8) a Baseline Model Heuristic Evaluation. In various embodiments, this aspect describes the method(s) by which measurements resulting from application of a Secondary measurement system to the specific specimen of medical fluid are compared to the Baseline Dataset, in order to produce a determination of the absence or incidence of material state change and associated potency loss in the specific specimen of medical fluid, and whether that specific specimen of medical fluid retains its specified properties, to a sufficient extent, to continue being considered as acceptable for patient administration. The distributed system may include (9) a Freeze-Damage Tracking System. In various embodiments, this aspect describes a system to track specific specimens of medical fluids that have indicated unacceptable material state change such that the causes of such change during storage, transport, and handling can be identified and remedied to prevent similar events in the future.

It shall be appreciated that FIG. 15 also represents a series of steps to be performed as a method of operation. For example, FIG. 15 represents a flow chart illustrating the relationships between the systems described in the distributed system directly above. In a first step, a medical fluid is produced by a manufacturer (1). In a second step, a representative sample of the medical fluid is extracted (2). In a third step, the representative sample is tested in a primary measurement system (3). In various embodiments, this testing specifies the (potency and/or material state) correlated to measurable properties of the medical fluid. In a fourth step, this information may be included in the Baseline Dataset, for determining the acceptable measurement range for any other specimen of that medical fluid (4). In a fifth step, this information may be made available via the baseline dataset transmission system (5). In various embodiments, this information may include the identity of the medical fluid and applied measurement parameters. Furthermore, this information may be transmitted and the medical fluid, from which the representative sample was obtained, ultimately intended for distribution to treat, or prevent disease, is packaged, and distributed according to said manufacturer's pre-existing protocols. In a sixth step, and at any point prior to patient administration, a specific specimen of medical fluid can be selected from the packages intended for distribution and administration (6). In a seventh step, this specific specimen can be measured at its location with a secondary measurement system (7) designed to operate along equivalent principles to the primary measurement system. When the result of the measurement of the specific specimen is obtained, the corresponding result from the primary measurement system on the representative sample and the corresponding Baseline Dataset, of which it is an element, can be obtained from the baseline dataset transmission system. In an eighth step, the baseline model heuristic evaluation method (8) can then be used to determine whether the specific specimen of medical fluid is to be considered to have retained its specified properties to a sufficient extent to proceed with distribution and patient administration. If so, then the specific specimen can continue to be stored, distributed, or administered. If not, in a ninth step the specific specimen may be marked as wastage with a record produced indicating the time, location, and identity of the specific specimen to be made available to the freeze-damage tracking system (9) for the purpose of ensuring traceability and auditability in the medical fluid storage and distribution process.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

What is claimed is:

1. A method for evaluating potency-correlated material states of a state dependent pharmaceutical product, comprising:
creating a data representation comprising a plurality of material state specifications for a pharmaceutical product using data gathered from at least one sensor acting on the pharmaceutical product, the material state specifications including a maximum allowable material state change;
correlating a minimum viable potency of the pharmaceutical product to the maximum allowable material state change;
communicating the data representation to at least one participant of a supply chain of the pharmaceutical product;
generating a specimen representation of a material state of a sample of the pharmaceutical product using data gathered from at least one sensor acting on the pharmaceutical product sample;
evaluating the specimen representation of the material state of the sample; and
determining whether the specimen representation of the material state of the sample exhibits a material state change greater than the maximum allowable material state change of the material state specifications.

2. The method of claim 1, further comprising:
discarding at least one pharmaceutical product if the specimen representation of the material state of the sample exhibits a material state change greater than the maximum allowable material state change of the material state specifications,
wherein the at least one pharmaceutical product is associated with a corresponding localized supply of the sample of the pharmaceutical product.

3. The method of claim 2, further comprising:
communicating identification data of the at least one pharmaceutical product to be discarded to at least one participant of the supply chain of the pharmaceutical product.

4. The method of claim 1, further comprising:
accepting at least one pharmaceutical product for use if the specimen representation of the material state of the sample exhibits a material state change less than the maximum allowable material state change of the material state specifications,
wherein the at least one pharmaceutical product for use is associated with a corresponding localized supply of the sample of the pharmaceutical product.

5. The method of claim 4, further comprising:
communicating identification data of the at least one pharmaceutical product to at least one participant of the supply chain of the pharmaceutical product.

6. The method of claim 1, further comprising:
determining an estimated potency of the sample of the pharmaceutical product; and
communicating the estimated potency of the sample to at least one participant of the supply chain of the pharmaceutical product.

7. The method of claim 1, wherein the evaluating the specimen representation of the material state of the sample step further comprises:
inputting at least one data component of the specimen representation of the material state of the sample of the pharmaceutical product into a neural network,
wherein the neural network comprises a trained machine learning model.

8. The method of claim 7, further comprising:
categorizing, by the trained machine learning model, whether the specimen representation of the material state of the sample comprises an acceptable material state change or an unacceptable material state change.

9. The method of claim 7, further comprising:
training the machine learning model based on the data representation of the plurality of material state specifications for the pharmaceutical product.

10. The method of claim 1, further comprising:
communicating, at a local level, at least one communication indicative of whether (1) the specimen representation of the material state of the sample exhibits a material state change determined to be greater than the maximum allowable material state change of the material state specifications or (2) the specimen representation of the material state of the sample exhibits a material state change determined to be less than the maximum allowable material state change of the material state specifications,
wherein the communication is chosen from the group comprising: a visual communication, an auditory communication, and/or a haptic communication.

11. The method of claim 1, further comprising:
communicating, across at least one blockchain network, at least one communication indicative of whether (1) the specimen representation of the material state of the sample exhibits a material state change determined to be greater than the maximum allowable material state change of the material state specifications or (2) the specimen representation of the material state of the sample exhibits a material state change determined to be less than the maximum allowable material state change of the material state specifications,
wherein the communication comprises data corresponding to an evaluation outcome of the evaluating the specimen representation of the material state of the sample step.

12. The method of claim 11, wherein the at least one blockchain network is accessible by a plurality of different participants of the supply chain of the pharmaceutical product.

13. The method of claim 11, wherein the at least one blockchain network provides a permanent ledger for performing an audit.

14. The method of claim 11, wherein the at least one blockchain network is only accessible by a subset of permissioned participants of the supply chain of the pharmaceutical product.

15. The method of claim 1, wherein the evaluating the specimen representation of the material state of the sample step further comprises evaluating the extent of chemical or conformational change in the pharmaceutical product.

16. The method of claim 1, wherein the evaluating the specimen representation of the material state of the sample step further comprises evaluating the extent of phase separation in the pharmaceutical product.

17. The method of claim 16, wherein the phase separation is chosen from the group comprising: agglomeration, flocculation, coalescence, creaming, and/or Ostwald ripening.

18. The method of claim 1, wherein the pharmaceutical product is a vaccine developed for human and/or animal use.

19. The method of claim 18, wherein the evaluating the specimen representation of the material state of the sample step further comprises evaluating the extent of change in the conformation of antigens.

20. The method of claim 1, wherein the pharmaceutical product is a vaccine comprising an adjuvant.

21. The method of claim 20, wherein the evaluating the specimen representation of the material state of the sample step further comprises evaluating the extent of phase separation of the coordinated antigen-to-adjuvant vaccine components.

22. The method of claim 20, wherein the evaluating the specimen representation of the material state of the sample step further comprises evaluating the extent of phase separation of the non-coordinated adjuvant vaccine components.

23. The method of claim 20, wherein the adjuvant comprises an oil and water emulsion.

24. The method of claim 1, wherein the pharmaceutical product is a vaccine component material.

25. The method of claim 1, wherein the pharmaceutical product is a vaccine component comprising an adjuvant.

26. The method of claim 1, wherein the creating the data representation comprising the plurality of material state specifications for a pharmaceutical product further comprises:
utilizing a phase contrast microscopy process to evaluate a size of the agglomerates.

27. The method of claim 1, wherein the evaluating the specimen representation of the material state of the sample further comprises evaluating a size of the agglomerates.

28. The method of claim 1, wherein the at least one sensor comprises a 2-D optical detector.

29. A system for evaluating potency-correlated material states of a state dependent pharmaceutical product, comprising:
a processor in communication with at least one non-transitory memory cell storing computer executable instructions thereon that when executed by the processor are configured to:
generate a data representation comprising a plurality of material state specifications for a pharmaceutical product using data gathered from at least one sensor acting on the pharmaceutical product, the material state specifications including a maximum allowable material state change;
correlate a minimum viable potency of the pharmaceutical product to the maximum allowable material state change;
communicate the data representation to at least one participant of a supply chain of the pharmaceutical product;
receive a data representation comprising a plurality of material state specifications for a pharmaceutical product, the material state specifications including a maximum allowable material state change;
generate a specimen representation of a material state of a sample of the pharmaceutical product using data gathered from at least one sensor acting on the pharmaceutical product sample;
utilize a machine learning model to evaluate the specimen representation of the material state of the sample in order to determine whether the specimen representation of the material state of the sample exhibits a material state change greater than the maximum allowable material state change of the material state specifications; and
communicate the specimen representation of the material state of the sample and its evaluation to at least one participant of a supply chain of the pharmaceutical product.

30. A computer program product, the computer program product being embodied in a non-transitory computer readable storage medium and comprising computer instructions for:
generating a data representation comprising a plurality of material state specifications for a pharmaceutical product using data gathered from at least one sensor acting on the pharmaceutical product, the material state specifications including a maximum allowable material state change;
correlating a minimum viable potency of the pharmaceutical product to the maximum allowable material state change;
communicating the data representation to at least one participant of a supply chain of the pharmaceutical product;
receiving a data representation comprising a plurality of material state specifications for a pharmaceutical product, the material state specifications including a maximum allowable material state change;
generating a specimen representation of a material state of a sample of the pharmaceutical product using data gathered from at least one sensor acting on the pharmaceutical product sample;
utilizing a machine learning model to evaluate the specimen representation of the material state of the sample in order to determine whether the specimen representation of the material state of the sample exhibits a material state change greater than the maximum allowable material state change of the material state specifications; and
communicating the specimen representation of the material state of the sample and its evaluation to at least one participant of a supply chain of the pharmaceutical product.
